(19) United States
(12) Patent Application Publication
(10) Pub. No.: US 2026/0358355 A1
(43) Pub. Date: Nov. 12, 2026

(54) IDENTIFYING RISK OF AND TREATING STROKE-INDUCED COGNITIVE IMPAIRMENT FOLLOWING THROMBECTOMY

(71) Applicant: University of Kentucky Research Foundation, Lexington, KY (US)

(72) Inventors: Keith R. Pennypacker, Lexington, KY (US); Justin F. Fraser, Lexington, KY (US); Christopher McLouth, Lexington, KY (US); Jordan P. Harp, Lexington, KY (US); Sarah Messmer, Lexington, KY (US); Jacqueline A. Frank, Lexington, KY (US)

(21) Appl. No.: 19/747,041

(22) Filed: Jul. 20, 2026

Related U.S. Application Data
(63) Continuation of application No. 18/227,192, filed on Jul. 27, 2023.
(60) Provisional application No. 63/393,016, filed on Jul. 28, 2022.

(51) Int. Cl. G01N 33/68; A61K 38/26; A61P 25/28

(52) U.S. Cl. CPC G01N 33/6896; A61K 38/26; A61P 25/28; G01N 2333/4745; G01N 2800/2814; G01N 2800/52

(57) ABSTRACT

Tools for identifying risk of and for treating vascular contributions to cognitive impairment and dementia (VCID) and stroke-induced cognitive impairment in a subject are provided and include a method and a device that involve detecting target proteins in systemic blood sample from the subject.

### RELATED APPLICATIONS

This application is a continuation of U.S. patent application Ser. No. 18/227,192 filed Jul. 27, 2026, which claims priority from U.S. Provisional Application Ser. No. 63/393,016 filed Jul. 28, 2022, the entire disclosures of which is incorporated herein by this reference.

### GOVERNMENT INTEREST

This invention was made with government support under grant number COBRE P30GM127211 awarded by the National Institutes of Health. The government has certain rights in the invention.

### TECHNICAL FIELD

The presently-disclosed subject matter generally relates to predicting stroke-induced cognitive impairment in a subject, and treating, including protecting against, stroke-induced cognitive impairment in a subject undergoing mechanical thrombectomy.

### INTRODUCTION

Emergent large vessel occlusion (ELVO) stroke is one of the leading causes of dementia and disability [19]. For ELVO candidates, endovascular mechanical thrombectomy (MT) has been shown to improve both neurological and cognitive functions in patients when compared to subjects treated with medical therapy alone [33]. However, despite effective strategies to re-establish blood flow, patients still suffer from significant cognitive effects from the injury [15, 28].

In stroke patients, cognitive disability confers a poorer prognosis regarding functional outcomes as well as increased dependence on caregivers [22, 27, 30]. Vascular contribution to cognitive impairment and dementia (VCID), found in 25-30% of stroke patients, is a particularly devastating long-term outcome [12]. While no disease-modifying treatments exist for VCID, early detection may focus medical attention and allow for increased rehabilitation intensity.

Accordingly, there remains a need in the art for tools for use in predicting stroke-induced cognitive impairment in a subject, and for treating stroke-induced cognitive impairment in a subject undergoing mechanical thrombectomy.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

Disclosed herein are methods and devices for use in identifying risk of vascular contributions to cognitive impairment and dementia (VCID) and/or stroke-induced cognitive impairment in a subject. Also disclosed herein are methods for use in protecting against stroke-induced cognitive impairment in a subject undergoing a mechanical thrombectomy procedure.

The presently-disclosed subject matter includes a method of identifying risk of vascular contributions to cognitive impairment and dementia (VCID) and/or stroke-induced cognitive impairment in a subject. In some embodiments, the method involves obtaining a blood sample from the subject; detecting target proteins in the sample, wherein the target proteins include one or more of the proteins selected from the group consisting of ANG, ARTN, CCL11, CCL14, CCL20, CCL23, CDCP1, CHL1, COL18A1, COMP, EFEMP1, EN-RAGE, ICAM1, IGLC2, IL-1α, IL-4, IL-6, IL-17C, IL-20, IL-24, IL-33, LTBP2, MCP-1, MCP-2, MCP-3, NRP1, TIMP1, TNC, APOM, CA4, CFHR5, CRTAC1, CXCL11, DNER, DPP4, ENG, F7, FAP, FGF-5, GPIBA, IGFBP3, IL7, IL7R, KIT, LILRB5, MEGF9, MFAP5, NCAM1, NOTCH1, NRTN, OSMR, PLTP, PRCP, PROC, PTPRS, QPCT, SCF, SELL, SERPINA7, TGFBI, TIE1, TNFSF14, and TWEAK; and predicting a negative effect on cognitive recovery when ANG, ARTN, CCL11, CCL14, CCL20, CCL23, CDCP1, CHL1, COL18A1, COMP, EFEMP1, EN-RAGE, ICAM1, IGLC2, IL-1α, IL-4, IL-6, IL-17C, IL-20, IL-24, IL-33, LTBP2, MCP-1, MCP-2, MCP-3, NRP1, TIMP1, or TNC is detected in the sample, and predicting a positive effect on cognitive recovery when APOM, CA4, CFHR5, CRTAC1, CXCL11, DNER, DPP4, ENG, F7, FAP, FGF-5, GPIBA, IGFBP3, IL7, IL7R, KIT, LILRB5, MEGF9, MFAP5, NCAM1, NOTCH1, NRTN, OSMR, PLTP, PRCP, PROC, PTPRS, QPCT, SCF, SELL, SERPINA7, TGFBI, TIE1, TNFSF14, or TWEAK is detected in the sample. In some embodiments, the target proteins include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86 of the proteins.

In some embodiments, the blood sample comprises systemic blood from the subject. In some embodiments, the blood sample comprises intracranial blood from the subject.

In some embodiments, the method also involves isolating plasma from the sample for use in detecting the target proteins.

In some embodiments, subject is a stroke patient who has undergone mechanical thrombectomy (MT). In some embodiments, the blood sample is peripheral blood collected just proximal to a thrombus removed by the MT. In some embodiments, the method also involves obtaining a proximal sample comprising blood collected proximal to a thrombus in the subject, and a distal sample comprising blood collected distal to the thrombus in the subject.

In some embodiments, the method also involves detecting the target proteins in the proximal sample and the distal sample, and predicting cognitive impairment when there are increased amounts of the target proteins in the proximal sample as compared to the distal sample.

In some embodiments, the method also involves administering a treatment for cognitive impairment to the subject, when a negative effect on cognitive recovery is predicted. In some embodiments, the subject has emergent large vessel occlusion (ELVO). In some embodiments, the treatment comprises a s-DPP-4 or a DPP-4 inhibitor. In some embodiments, the inhibitor is gliptin, linagliptin, sitagliptin, vildagliptin, saxagliptin, gemigliptin, anagliptin, teneligliptin, alogliptin, trelagliptin, omarigliptin, evogliptin, gosogliptin, dutogliptin, or berberine,

In some embodiments, the method also involves obtaining a second blood sample from the subject, collected at a distinct time. In some embodiments, the method also involves comparing the detected target proteins in the sample to the detected target proteins in the second sample. In this regard, the method can be used to monitor risk over time, including identifying a trend of increasing risk or decreasing risk. For example, in some embodiments, when the subject has received treatment for cognitive impairment, such as a DPP-4 inhibitor, risk of cognitive impairment for the subject can be monitored over time, such that the impact of the treatment can be assessed.

In some embodiments, the method also involves using a device to detect the proteins in the sample, wherein the device comprises a combination of probes affixed to a substrate, comprising a probe specific for each of target proteins and additional target proteins. In some embodiments, the method also involves using a device to detect the proteins in the sample, wherein the device comprises a combination of probes affixed to a substrate, comprising a probe specific for each of the target proteins. In some embodiments, the device could be a microfluidic enzyme-linked immunosorbent assay (ELISA) device.

The presently-disclosed subject matter also includes a device for use in identifying risk of stroke-induced cognitive impairment in a subject, which includes a combination of probes, comprising a probe specific for each of the target proteins, wherein the target proteins include one or more of ANG, ARTN, CCL11, CCL14, CCL20, CCL23, CDCP1, CHL1, COL18A1, COMP, EFEMP1, EN-RAGE, ICAM1, IGLC2, IL-1α, IL-4, IL-6, IL-17C, IL-20, IL-24, IL-33, LTBP2, MCP-1, MCP-2, MCP-3, NRP1, TIMP1, TNC, APOM, CA4, CFHR5, CRTAC1, CXCL11, DNER, DPP4, ENG, F7, FAP, FGF-5, GPIBA, IGFBP3, IL7, IL7R, KIT, LILRB5, MEGF9, MFAP5, NCAM1, NOTCH1, NRTN, OSMR, PLTP, PRCP, PROC, PTPRS, QPCT, SCF, SELL, SERPINA7, TGFBI, TIE1, TNFSF14, and TWEAK. In some embodiments, the device is provided as a microfluidic ELISA device.

The presently-disclosed subject matter includes a method of protecting against stroke-induced cognitive impairment in a subject undergoing a mechanical thrombectomy procedure. In some embodiments, the method involves administering to the subject an effective amount of a s-DPP-4 or a DPP-4 inhibitor.

The presently-disclosed subject matter is also directed to the use of sDPP-4 or a DPP-4 inhibitor as adjuvant treatment to improve cognitive recovery for a subject undergoing a mechanical thrombectomy procedure.

In some embodiments of the method involving administration of s-DPP-4 or DPP-4 inhibitor, the subject has emergent large vessel occlusion (ELVO). In some embodiments, s-DPP-4 is administered. In some embodiments, a DPP-4 inhibitor is administered. The DPP-4 inhibitor can be, for example, a gliptin. The DPP-4 inhibitor can be, for example, linagliptin, sitagliptin, vildagliptin, saxagliptin, gemigliptin, anagliptin, teneligliptin, alogliptin, trelagliptin, omarigliptin, evogliptin, gosogliptin, dutogliptin, or berberine,

In some embodiments of the method involving administration of s-DPP-4 or DPP-4 inhibitor, the method also involves obtaining a blood sample from the subject and detecting target proteins in the sample, wherein the target proteins include a protein selected from the group consisting of: ANG, ARTN, CCL11, CCL14, CCL20, CCL23, CDCP1, CHL1, COL18A1, COMP, EFEMP1, EN-RAGE, ICAM1, IGLC2, IL-1α, IL-4, IL-6, IL-17C, IL-20, IL-24, IL-33, LTBP2, MCP-1, MCP-2, MCP-3, NRP1, TIMP1, TNC, APOM, CA4, CFHR5, CRTAC1, CXCL11, DNER, DPP4, ENG, F7, FAP, FGF-5, GPIBA, IGFBP3, IL7, IL7R, KIT, LILRB5, MEGF9, MFAP5, NCAM1, NOTCH1, NRTN, OSMR, PLTP, PRCP, PROC, PTPRS, QPCT, SCF, SELL, SERPINA7, TGFBI, TIE1, TNFSF14, and TWEAK.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-1B illustrate the inter-protein relatedness among proteins predictive of both MoCA at discharge (FIG. 1A) and MoCA at 90-days (FIG. 1B).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Because mechanical thrombectomy (MT) involves removing the thrombus with a stent retriever to open a blocked cerebral artery, the procedure allows for samples to be collected, including: distal blood within the artery immediately downstream from the clot, peripheral blood just proximal to the clot (systemic arterial blood in the cervical carotid artery), and the thrombus itself upon removal from the human subject.

From this technique, a prospective tissue bank, the Blood And Clot Thrombectomy Registry and Collaboration (BACTRAC; NCT03153683) has been established. The analysis of distal blood shows the initial pathophysiological reaction to the occlusion, which stimulates a systemic response to the ischemic injury. Prior to MT, researchers relied primarily on cell culture models to determine the cellular response to ischemia. Now, the molecular changes within the tissues of a stroke patient can be observed. The protocol separates patient blood into plasma for protein analysis and leukocytes for the isolation of mRNA subsequent analysis of gene and protein expression.

Subject demographics and disease-related clinical variables are also collected. Clinical data points on each subject are entered into the REDCap (Research Electronic Data Capture) database. The following demographic and clinical data are being captured: 1) age, 2) sex, 3) race/ethnicity, 4) body mass index, 5) premorbid Modified Rankin Score (mRS), 6) location of the thrombus (e.g. M1 segment of middle cerebral artery), 6) likely source of the thrombus (cardioembolic, intracranial stenosis, dissection, carotid occlusion, infection, unknown), 7) presence of tandem occlusion, 8) time from Last Known Normal to thrombectomy completion (reopening of vessel), 9) thrombectomy success as rated by TICI score, 7) NIHSS on admission and prior to discharge, 10) Mini-Montreal Cognitive Assessment prior to discharge, 11) medical co-morbidities including hypertension, diabetes and hypercholesterolemia, 12) premorbid modified Rankin Scale, and 13) administration of intravenous tPA.

Patients with ELVO are now often treated with mechanical thrombectomy (MT), but some patients suffer from stroke-induced cognitive impairment. There is a clear unmet need of predictive tools that could guide early clinical intervention to provide better cognitive prognosis for ELVO patients. There is also a clear unmet need for treatments that could improve cognitive outcomes for ELVO patients.

Disclosed herein are methods and devices for use in identifying risk of vascular contributions to cognitive impairment and dementia (VCID) and/or stroke-induced cognitive impairment in a subject. These methods and devices can be used, for example, for real-time analysis in hospitals. These methods and devices address an unmet medical need and will improve the prognosis of stroke patients at risk for stroke-induced dementia.

Also disclosed herein are methods for use in protecting against stroke-induced cognitive impairment in a subject undergoing a mechanical thrombectomy procedure. Soluble dipeptidyl peptidase-4 (sDPP-4) was found to be significantly increased in systemic blood in patients that performed better on cognitive tests. Additionally, sDPP4 expression was significantly correlated with improving cognition. The present inventors' data indicate that administration of sDPP-4 or inhibitors of DPP-4 (which increase sDPP-4 levels) during a thrombectomy procedure would improve cognitive outcomes of stroke patients. Because stroke is a major cause of dementia, the methods address an unmet medical need as an adjuvant therapy for stroke patients undergoing thrombectomy to improve cognitive and other stroke-related outcomes.

The presently-disclosed subject matter includes a method of identifying risk of vascular contributions to cognitive impairment and dementia (VCID) and/or stroke-induced cognitive impairment in a subject.

In some embodiments, the method involves obtaining a blood sample from the subject, detecting target proteins in the sample, wherein the target proteins include one or more of the proteins set forth in Table 1.

TABLE 1

| Predictive of Negative Effect | Predictive of Positive Effect |
|---|---|
| Angiogenin (ANG), | Apolipoprotein M (APOM), |
| Artemin (ARTN), | Carbonic Anhydrase 4 (CA4), |
| Eotaxin (CCL11), | Complement Factor H-Related Protein 5 |
| Chemokine (C-C motif) ligand 14 (CCL14), | (CFHR5), |
| Chemokine (C-C motif) ligand 20 (CCL20), | Cartilage Acidic Protein 1 (CRTAC1), |
| Chemokine (C-C motif) ligand 23 (CCL23), | C-X-C Motif Chemokine Ligand 11 (CXCL11), |
| CUB domain-containing protein 1 (CDCP1), | Delta and notch-like epidermal growth factor- |
| Close Homolog of L1 (CHL1), | related receptor (DNER), |
| Collagen Type XVIII Alpha 1 (COL18A1), | Soluble dipeptidyl peptidase-4 (s-DPP4), |
| Cartilage oligomeric matrix protein (COMP), | Endoglin (ENG), |
| EGF-containing fibulin-like extracellular matrix | Coagulation Factor VII (F7), |
| protein 1 (EFEMP1), | Prolyl endopeptidase (FAP), |
| Extracellular newly identified RAGE-binding | Fibroblast Growth Factor 5 (FGF-5), |
| protein (EN-RAGE), | Glycoprotein Ib Alpha (GP1BA), |
| Intercellular Adhesion Molecule 1 (ICAM1), | Insulin-like growth factor binding protein-3 |
| Immunoglobulin Lambda Constant 2 (IGLC2), | (IGFBP3), |
| Interleukin-1 alpha (IL-1α), | Interleukin 7 (IL7), |
| Interleukin-4 (IL-4), | Interleukin-7 receptor subunit alpha (IL7R), |
| Interleukin-6 (IL-6), | Mast/stem Cell Growth Factor Receptor Kit |
| Interleukin-17C (IL-17C), | (KIT), |
| Interleukin-20 (IL-20), | Leukocyte Immunoglobulin-Like Receptor |
| Interleukin-24 (IL-24), | Subfamily B Member 5 (LILRB5), |
| Interleukin-33 (IL-33), | Multiple epidermal growth factor-like domains |
| Latent-transforming growth factor beta-binding | protein 9 (MEGF9), |
| protein 2 (LTBP2), | Microfibrillar Associated Protein 5 (MFAP5), |
| Monocyte chemotactic protein-1 (MCP-1), | Neural Cell Adhesion Molecule 1 (NCAM1), |
| Monocyte chemotactic protein-2 (MCP-2), | Notch Receptor 1 (NOTCH1), |
| Monocyte chemotactic protein-3 (MCP-3), | Neurturin (NRTN), |
| Neuropilin-1 (NRP1), | Oncostatin M Receptor (OSMR), |
| Tissue Inhibitor of Metalloproteinase 1 (TIMP1), | Phospholipid Transfer Protein (PLTP), |
| and | Prolylcarboxypeptidase (PRCP), |
| Tenascin-C (TNC) | Protein C (PROC), |
| | Protein Tyrosine Phosphatase Receptor Type S |
| | (PTPRS), |
| | Glutaminyl-Peptide Cyclotransferase (QPCT), |
| | Stem cell factor (SCF), |
| | L-Selectin (SELL), |
| | Thyroxine-binding globulin (SERPINA7), |
| | Transforming growth factor-beta-induced |
| | protein ig-h3 (TGFBI), |
| | Tyrosine-protein kinase receptor tie-1 (TIE1), |
| | Tumor Necrosis Factor Ligand Superfamily |
| | Member 14 (TNFSF14), and |
| | Tumor Necrosis Factor-like Weak Inducer of |
| | Apoptosis (TWEAK) |

Table 1 includes proteins predictive of a negative effect on cognitive recovery in the left column, and proteins predictive of a positive effect on cognitive recovery in the right column. Accordingly, the presence of a biomarker protein from the left column (predictor of negative effect) in a blood sample from a subject would indicate an increased risk of cognitive impairment. Meanwhile, the presence of a biomarker protein from the right column (predictor of positive effect) in a blood sample from a subject would indicate a decreased risk of cognitive impairment.

In some embodiments, the method involves obtaining a blood sample from the subject; detecting target proteins in the sample, wherein the target proteins include one or more of the proteins selected from the group consisting of ANG, ARTN, CCL11, CCL14, CCL20, CCL23, CDCP1, CHL1, COL18A1, COMP, EFEMP1, EN-RAGE, ICAM1, IGLC2, IL-1α, IL-4, IL-6, IL-17C, IL-20, IL-24, IL-33, LTBP2, MCP-1, MCP-2, MCP-3, NRP1, TIMP1, TNC, APOM, CA4, CFHR5, CRTAC1, CXCL11, DNER, DPP4, ENG, F7, FAP, FGF-5, GPIBA, IGFBP3, IL7, IL7R, KIT, LILRB5, MEGF9, MFAP5, NCAM1, NOTCH1, NRTN, OSMR, PLTP, PRCP, PROC, PTPRS, QPCT, SCF, SELL, SERPINA7, TGFBI, TIE1, TNFSF14, and TWEAK; and predicting a negative effect on cognitive recovery when ANG, ARTN, CCL11, CCL14, CCL20, CCL23, CDCP1, CHL1, COL18A1, COMP, EFEMP1, EN-RAGE, ICAM1, IGLC2, IL-1α, IL-4, IL-6, IL-17C, IL-20, IL-24, IL-33, LTBP2, MCP-1, MCP-2, MCP-3, NRP1, TIMP1, or TNC is detected in the sample, and predicting a positive effect on cognitive recovery when APOM, CA4, CFHR5, CRTAC1, CXCL11, DNER, DPP4, ENG, F7, FAP, FGF-5, GP1BA, IGFBP3, IL7, IL7R, KIT, LILRB5, MEGF9, MFAP5, NCAM1, NOTCH1, NRTN, OSMR, PLTP, PRCP, PROC, PTPRS, QPCT, SCF, SELL, SERPINA7, TGFBI, TIE1, TNFSF14, or TWEAK is detected in the sample. In some embodiments, the target proteins include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or 86 of the proteins.

In some embodiments, of the method the target proteins include one or more of the proteins selected from the group consisting of APOM, DNER, s-DPP4, IGFBP3, MEGF9, and SERPINA7, and further involve predicting a positive effect on cognitive recovery when APOM, DNER, DPP4, IGFBP3, MEGF9, or SERPINA7 is detected in the sample;

In some embodiments, of the method the target proteins include one or more of the proteins selected from the group consisting of include one or more of APOM, DNER, FAP, IGFBP3, SCF, and TGFBI, and further involve predicting a positive effect on cognitive recovery when APOM, DNER, FAP, IGFBP3, SCF, or TGFBI is detected in the sample;

In some embodiments, of the method the target proteins include one or more of the proteins selected from the group consisting of include one or more of ARTN, CCL11, COMP, MCP-1, MCP-2, and NRP1, and further involve predicting a negative effect on cognitive recovery when ARTN, CCL11, COMP, MCP-1, MCP-2, or NRP1 is detected in the sample;

In some embodiments, of the method the target proteins include one or more of the proteins selected from the group consisting of include one or more of IGFBP3, MCP-2, ARTN, TIE1, IL7R, and LTBP2, and further involve predicting a negative effect on cognitive recovery when MCP-2, ARTN, or LTBP2 is detected in the sample, and predicting a positive effect on cognitive recovery when IGFBP3, TIE1, IL7R, or LTBP2 is detected in the sample;

In some embodiments, of the method the target proteins include one or more of the proteins selected from the group consisting of include one or more of DPP4, CCL11, IGFBP3, DNER, NRP1, MCP1, and COMP, and further involve predicting a negative effect on cognitive recovery when CCL11, NRP1, MCP1, or COMP is detected in the sample, and predicting a positive effect on cognitive recovery when DPP4, IGFBP3, or DNER is detected in the sample;

In some embodiments, of the method the target proteins include one or more of the proteins selected from the group consisting of include one or more of CCL20, CRTAC1, DNER, DPP4, FAP, PTPRS, and TNC, and further involve predicting a negative effect on cognitive recovery when CCL20 or TNC is detected in the sample, and predicting a positive effect on cognitive recovery when CRTAC1, DNER, DPP4, FAP, or PTPRS is detected in the sample; or

In some embodiments, of the method the target proteins include one or more of the proteins selected from the group consisting of include one or more of DNER, APOM, IGFBP3, SCF, FAP, and DPP4, and further involve predicting a positive effect on cognitive recovery when DNER, POM, IGFBP3, SCF, FAP, or DPP4 is detected in the sample.

In some embodiments, of the method the target proteins include IGFBP3 and further involve predicting a positive effect on cognitive recovery IGFBP3 is detected in the sample.

In some embodiments, the blood sample comprises systemic blood from the subject. In some embodiments, the blood sample comprises intracranial blood from the subject.

In some embodiments, the method also involves isolating plasma from the sample for use in detecting the target proteins.

In some embodiments, subject is a stroke patient who has undergone mechanical thrombectomy (MT). In some embodiments, the blood sample is peripheral blood collected just proximal to a thrombus removed by the MT. In some embodiments, the method also involves obtaining a proximal sample comprising blood collected proximal to a thrombus in the subject, and a distal sample comprising blood collected distal to the thrombus in the subject.

In some embodiments, the method also involves detecting the target proteins in the proximal sample and the distal sample, and predicting cognitive impairment when there are increased amounts of the target proteins in the proximal sample as compared to the distal sample.

In some embodiments, the method also involves administering a treatment for cognitive impairment to the subject, when a negative effect on cognitive recovery is predicted. In some embodiments, the subject has emergent large vessel occlusion (ELVO). In some embodiments, the treatment comprises s-DPP-4 or a DPP-4 inhibitor. In some embodiments, the inhibitor is gliptin, linagliptin, sitagliptin, vildagliptin, saxagliptin, gemigliptin, anagliptin, teneligliptin, alogliptin, trelagliptin, omarigliptin, evogliptin, gosogliptin, dutogliptin, or berberine,

In some embodiments, the method also involves obtaining a second blood sample from the subject, collected at a distinct time. In some embodiments, the method also involves comparing the detected target proteins in the sample to the detected target proteins in the second sample. In this regard, the method can be used to monitor risk over time, including identifying a trend of increasing risk or decreasing risk. For example, in some embodiments, when the subject has received treatment for cognitive impairment, such as a DPP-4 inhibitor, risk of cognitive impairment for the subject can be monitored over time, such that the impact of the treatment can be assessed.

In some embodiments, the method also involves using a device to detect the proteins in the sample, wherein the device comprises a combination of probes affixed to a substrate, comprising a probe specific for each of target proteins and additional target proteins. In some embodiments, the method also involves using a device to detect the proteins in the sample, wherein the device comprises a combination of probes affixed to a substrate, comprising a probe specific for each of the target proteins. In some embodiments, the device could be a microfluidic enzyme-linked immunosorbent assay (ELISA) device.

The presently-disclosed subject matter also includes a device for use in identifying risk of stroke-induced cognitive impairment in a subject, which includes a combination of probes, comprising a probe specific for each of the target proteins, wherein the target proteins include one or more of ANG, ARTN, CCL11, CCL14, CCL20, CCL23, CDCP1, CHL1, COL18A1, COMP, EFEMP1, EN-RAGE, ICAM1, IGLC2, IL-1α, IL-4, IL-6, IL-17C, IL-20, IL-24, IL-33, LTBP2, MCP-1, MCP-2, MCP-3, NRP1, TIMP1, TNC, APOM, CA4, CFHR5, CRTAC1, CXCL11, DNER, DPP4, ENG, F7, FAP, FGF-5, GPIBA, IGFBP3, IL7, IL7R, KIT, LILRB5, MEGF9, MFAP5, NCAM1, NOTCH1, NRTN, OSMR, PLTP, PRCP, PROC, PTPRS, QPCT, SCF, SELL, SERPINA7, TGFBI, TIE1, TNFSF14, and TWEAK. In some embodiments, the device is provided as a microfluidic ELISA device.

The presently-disclosed subject matter includes a method of protecting against stroke-induced cognitive impairment in a subject undergoing a mechanical thrombectomy procedure. In some embodiments, the method involves administering to the subject an effective amount of s-DPP-4 or a DPP-4 inhibitor.

The presently-disclosed subject matter is also directed to the use of sDPP-4 or a DPP-4 inhibitor as adjuvant treatment to improve cognitive recovery for a subject undergoing a mechanical thrombectomy procedure.

In some embodiments of the method involving administration of s-DPP-4 or DPP-4 inhibitor, the subject has emergent large vessel occlusion (ELVO). In some embodiments, s-DPP-4 is administered. In some embodiments, a DPP-4 inhibitor is administered. The DPP-4 inhibitor can be, for example, a gliptin. The DPP-4 inhibitor can be, for example, linagliptin, sitagliptin, vildagliptin, saxagliptin, gemigliptin, anagliptin, teneligliptin, alogliptin, trelagliptin, omarigliptin, evogliptin, gosogliptin, dutogliptin, or berberine,

In some embodiments of the method involving administration of s-DPP-4 or DPP-4 inhibitor, the method also involves obtaining a blood sample from the subject and detecting target proteins in the sample, wherein the target proteins include a protein selected from the group consisting of: ANG, ARTN, CCL11, CCL14, CCL20, CCL23, CDCP1, CHL1, COL18A1, COMP, EFEMP1, EN-RAGE, ICAM1, IGLC2, IL-1α, IL-4, IL-6, IL-17C, IL-20, IL-24, IL-33, LTBP2, MCP-1, MCP-2, MCP-3, NRP1, TIMP1, TNC, APOM, CA4, CFHR5, CRTAC1, CXCL11, DNER, DPP4, ENG, F7, FAP, FGF-5, GPIBA, IGFBP3, IL7, IL7R, KIT, LILRB5, MEGF9, MFAP5, NCAM1, NOTCH1, NRTN, OSMR, PLTP, PRCP, PROC, PTPRS, QPCT, SCF, SELL, SERPINA7, TGFBI, TIE1, TNFSF14, and TWEAK.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11 (9): 1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Introduction to Examples 2-7

As described herein, human biospecimens obtained from ELVO stroke subjects treated with mechanical thrombectomy (MT) were tested. The Blood And Clot Thrombectomy Registry And Collaboration (BACTRAC) protocol (clinicaltrials.gov; NCT 03153683) allows for processing of both intracranial (distal to thrombus) and systemic (carotid) arterial blood samples. Systemic samples are used as acquiring systemic arterial blood is an easier prognostic step than intracranial; however, intracranial blood samples allow for comparison of systemic protein expression with expression at the site of infarction. Using these data, inflammatory-associated proteomic responses have been reported, which are predictive of clinical outcomes, such as functional recovery.

Several biomarkers (total tau, pTau181, Aβ40, Aβ42, Aβ42/40), GFAP, and Nfl) of dementia also have been reported to be associated with VCID [3, 4, 13, 25, 31]. However, because stroke is an acute vascular event not always present in ADRD populations, the addition of other potential biomarkers will strengthen the predictive model for determining stroke patients most likely to incur cognitive impairment. The objective of this study was to utilize the BACTRAC registry to identify proteomic biomarkers predictive of cognitive performance at discharge and 90-days in ELVO subjects treated with MT.

### Example 2: Tissue Sample and Clinical Data Acquisition

This study utilizes the BACTRAC tissue registry (clinicaltrials.gov; NCT 03153683) of human biospecimens acquired during ELVO stroke in subjects undergoing MT. This study is approved by the University of Kentucky Institutional Review Board (IRB). Inclusion criteria for this study included all ELVO subjects who were candidates for MT and aged 18 or older. Exclusion criteria for this study included age less than 18, subjects who were pregnant, incarcerated subjects, and subjects unable to consent within the IRB-outlined 72-h window. Subjects included in this current study were enrolled between Jun. 21, 2017, and Mar. 1, 2021. Methods of acquiring systemic blood during MT per the BACTRAC protocol has been previously published.8 Briefly, arterial blood proximal to the clot is sampled immediately prior to recanalization. Blood is aliquoted into BD Microtainer tubes with K2E (K2EDTA; Becton, Dickinson and Company) and spun down at 2000 rcf for 15 min, plasma is promptly extracted off the top and flash frozen on dry ice in a Wheaton CryoELITE cryogenic vial (DWK Life Sciences; Millville, New Jersey). Samples are stored at −80 C until batches are sent to Olink Proteomics (Olink Proteomics, Boston, MA) for analysis of plasma protein. OLINK proteomics is a high-throughput, multiplexed protein analysis technology that allows for the simultaneous quantification of hundreds of proteins in a single sample.

While BACTRAC enrollment continues, the dataset was limited to this interval to ensure complete primary outcomes. Clinical data are collected on each subject including demographics, comorbidities, relevant labs, radiographic outcome, thrombectomy outcome, and both functional and cognitive outcome metrics. Specific to this study, Montreal Cognitive Assessment (MoCA) scores were either clinically documented in the full 30-point scale or the abbreviated mini-MoCA, 12-point scale. The Montreal Cognitive Assessment (MoCA) is a widely used screening tool to assess cognitive function in adults. It was developed to detect mild cognitive impairment (MCI) and early dementia. The test measures different cognitive domains, such as attention, memory, language, orientation, visuospatial skills, and executive function. The full MoCA test consists of 30 questions and takes approximately 10-15 min to administer. It assesses a wide range of cognitive functions and is sensitive to mild cognitive impairment. The full MoCA test is typically administered by a trained healthcare professional, such as a physician, nurse, or psychologist. The mini-MoCA is a shorter version of the MoCA test, consisting of only 12 questions, and it takes approximately 5-10 min to administer. The mini-MoCA is a quicker and more convenient screening tool for busy healthcare professionals or for use in settings where time is limited. It focuses on the most critical cognitive domains, such as attention, memory, and executive function. A limitation of this study is inconsistent administration of the MoCA or mini-MoCA at discharge by the medical professionals. To remedy this inconsistency in scoring the mini-MoCA score was converted into a 30-point scale for comparability using the following equation.

Pro-rated Score=(30×mini-MoCA score)/12

### Example 3: Specimen Processing and Proteomic Analysis

Methods for biospecimen processing for proteomic analysis has been previously published [8, 16, 17, 18, 26]. Plasma samples are sent to Olink Proteomics (Olink Proteomics, Boston, MA) for analysis of 96 cardiometabolic and 96 inflammatory proteins. Olink returns proteomic expression values in a Normalized Protein expression (NPX) value, which is in log 2 scale to reduce intra- and inter-assay variability when running statistics across sample sets. Presently, Olink has been included in over 11,000 publications (www.olink.com/).

### Example 4: Statistical Analysis

When assessing for the presence of comorbidities such as hypertension, hyperlipidemia, and diabetic status, unpaired t-tests were utilized. For categorical variables such as location of thrombus (left, right, basilar), and body mass index (BMI) (normal, overweight, and obese) ANOVA was utilized. For continuous variables such as infarct volume, age of patient, and infarct time, Pearson correlations were utilized. Protein concentrations from individual patient samples were assessed with MoCA score at discharge and 90-days using Pearson correlations. For all analyses, p≤0.05 was considered significant. Data analysis was performed in GraphPad Prism Version 9.3.1.

### Example 5: Subject Demographic and Comorbid Data

Table 2 demonstrates the demographic, comorbid, and outcome data for the separate cohorts of subjects analyzed in this study. There were n=52 subjects with discharge MoCA scores and n=28 subjects with 90-day MoCA scores.

TABLE 2
Demographic, comorbid, and outcome data.

| | MOCA at Discharge | | MOCA at 90 Days | |
|---|---|---|---|---|
| | Overall | Proteomic | Overall | Proteomic |
| | Cohort n = 52 | Cohort n = 23 | Cohort n = 28 | Cohort n = 13 |
| Age (median; range) | 62 ± 14.7 | 59 ± 15.1 | 60 ± 16.5 | 59 ± 18.7 |

Sex

| | | | | | | | | |
|---|---|---|---|---|---|---|---|---|
| Female | 29 | (56) | 15 | (65) | 10 | (36) | 5 | (38) |
| Male | 23 | (44) | 8 | (35) | 18 | (64) | 8 | (62) |

BMI

| | | | | | | | | |
|---|---|---|---|---|---|---|---|---|
| <18.5 | 2 | (4) | 1 | (4) | 0 | (0) | 0 | (0) |
| 18.5-24.9 | 10 | (19) | 5 | (22) | 5 | (18) | 2 | (15) |
| 25-29.9 | 15 | (29) | 8 | (35) | 9 | (32) | 6 | (46) |
| >30 | 25 | (48) | 9 | (39) | 14 | (50) | 5 | (38) |

Comorbidities

| | | | | | | | | |
|---|---|---|---|---|---|---|---|---|
| Hypertension | 42 | (81) | 18 | (78) | 23 | (82) | 11 | (85) |
| Diabetes Mellitus II | 14 | (27) | 2 | (9) | 6 | (21) | 2 | (15) |
| Hyperlipidemia | 16 | (31) | 5 | (22) | 8 | (29) | 4 | (31) |
| Atrial Fibrillation | 20 | (38) | 10 | (36) | 9 | (39) | 5 | (38) |
| Previous Stroke | 6 | (12) | 3 | (13) | 1 | (4) | 0 | (0) |

Smoking Status*

| | | | | | | | | |
|---|---|---|---|---|---|---|---|---|
| Never | 25 | (48) | 11 | (52) | 12 | (52) | 6 | (50) |
| Currently | 17 | (33) | 8 | (38) | 8 | (35) | 5 | (42) |
| Previously | 6 | (2) | 2 | (10) | 3 | (13) | 1 | (8) |
| (>6 months) | | | | | | | | |

National institute of health stroke scale/score (NIHSS) on Admission

| | | | | | | | | |
|---|---|---|---|---|---|---|---|---|
| Minor Stroke (1-4) | 3 | (6) | 1 | (4) | 2 | (7) | 1 | (8) |
| Moderate Stroke (5-15) | 32 | (63) | 17 | (74) | 15 | (54) | 7 | (54) |
| Moderate/Severe (16-20) | 10 | (19) | 4 | (17) | 7 | (25) | 4 | (30) |
| Severe Stroke (≥21) | 6 | (12) | 1 | (4) | 4 | (14) | 1 | (8) |

NIHSS at Discharge*

| | | | | | | | | |
|---|---|---|---|---|---|---|---|---|
| Minor Stroke (1-4) | 38 | (74) | 16 | (70) | 16 | (59) | 6 | (50) |
| Moderate Stroke (5-15) | 12 | (24) | 6 | (26) | 10 | (37) | 5 | (42) |
| Moderate/Severe (16-20) | 1 | (2) | 1 | (4) | 1 | (4) | 1 | (8) |
| Severe Stroke (≥21) | 0 | (0) | 0 | (0) | 0 | (0) | 0 | (0) |

Thrombolysis in cerebral infarction (TICI) Score

| 2A = <50% Perfusion | 0 | (0) | 0 | (0) | 0 | (0) | 0 | (0) |
|---|---|---|---|---|---|---|---|---|
| 2B = >50% Perfusion | 19 | (37) | 9 | (39) | 11 | (44) | 6 | (46) |
| 3 = Full Perfusion | 32 | (63) | 14 | (61) | 14 | (56) | 7 | (54) |
| LKN to | 564 ± 356 | | 657 ± 405 | | 625 ± 402 | | 796 ± 460 | |
| Thrombectomy | | | | | | | | |

| | | | | |
|---|---|---|---|---|
| Infarct Volume (mm³) | 22,353 ± 27,183 | 15,991 ± 18,015 | 32,808 ± 37,961 | 27,403 ± 36,709 |

Location of Thombus

| | | | | | | | | |
|---|---|---|---|---|---|---|---|---|
| Left ICA | 2 | (4) | 1 | (4) | 1 | (4) | 1 | (8) |
| Right ICA | 1 | (2) | 0 | (0) | 0 | (0) | 0 | (0) |
| Left MCA | 16 | (30) | 6 | (26) | 8 | (28) | 1 | (8) |
| Right MCA | 28 | (54) | 14 | (61) | 14 | (50) | 9 | (69) |
| Basilar | 5 | (10) | 2 | (9) | 5 | (18) | 2 | (15) |

Source of Thrombus

| | | | | | | | | |
|---|---|---|---|---|---|---|---|---|
| Cardioembolic | 32 | (61) | 14 | (59) | 15 | (53) | 7 | (53) |
| Atherembolic | 14 | (27) | 3 | (13) | 7 | (25) | 2 | (15) |
| Intracranial Stenosis | 1 | (2) | 1 | (5) | 1 | (5) | 1 | (8) |
| Dissection | 1 | (2) | 1 | (5) | 1 | (5) | 1 | (8) |
| Carotid Occlusion | 1 | (2) | 1 | (5) | 1 | (5) | 1 | (8) |
| Infection | 0 | (0) | 0 | (0) | 0 | (0) | 0 | (0) |
| Unknown | 3 | (6) | 3 | (13) | 2 | (7) | 1 | (8) |

CTA Collateral Score*

| | | | | | | | | |
|---|---|---|---|---|---|---|---|---|
| 0 | 2 | (9) | 2 | (10) | 4 | (24) | 3 | (25) |
| 1 | 15 | (68) | 14 | (70) | 10 | (58) | 7 | (58) |
| 2 | 3 | (14) | 2 | (10) | 2 | (12) | 2 | (17) |
| 3 | 2 | (9) | 2 | (10) | 1 | (6) | 0 | (0) |

| | | | |
|---|---|---|---|
| *missing 1 | *missing 2 | *missing 5 | *missing 1 |
| (n = 51) | (n = 23) | (n = 23) | (n = 12) |
| **missing 3 | **missing 1 | **missing 1 | |
| (n = 49) | (n = 24) | (n = 27) | |
| ***missing 7 | ***missing 3 | ***missing 3 | |
| (n = 45) | (n = 22) | (n = 25) | |
| ****missing 10 | | ****missing 9 | |
| (n = 42) | | (n = 19) | |
| | | *****missing 11 | |
| | | (n = 17) | |

### Example 6: MoCA Scores and Subject Characteristics

Discharge MoCA scores were assessed in relation to demographic data, comorbidities, and outcome. When location of thrombus was assessed, subjects with a basilar thrombus were found to have significantly lower discharge MoCA scores when compared to the right-sided thrombus group (p=0.01) indicating greater cognitive burden in basilar subjects. No other thrombus location assessments were significant, including no relationship between left-vs. right-sided locations. There was a positive correlation between low-density lipoprotein (LDL) and MoCA score at discharge (p=0.02; R2=0.12). There were no significant relationships between discharge MoCA scores and patient age, sex, presence of hypertension, hyperlipidemia, diabetes diagnosis, BMI, Alc, TSH levels at presentation, high-density lipoprotein (HDL), triglyceride level, total cholesterol, previous stroke, infarct time, infarct volume, or whether subject received tPA prior to MT.

90-day MoCA scores were also assessed in relation to demographic data, comorbidities, and outcome. Age was found to have a negative correlation with 90-day MoCA scores, indicating older subjects performed worse on the cognitive examination (p=0.03). When subjects were broken down into respective BMI categories (<24.9 as normal, 25-29.9 as overweight, and >30 as obese), ANOVA testing revealed the normal weighted group had significantly lower 90-day MoCA scores when compared to the obese group (p=0.03). There were no significant relationships identified when assessing 90-day MoCA scores and patient sex, A1c, TSH, LDL, HDL, triglycerides, total cholesterol; diagnosis of hyperlipidemia, hypertension, diabetes; infarct time, infarct volume, atrial fibrillation or location/source of thrombus.

### Example 6: MoCA Scores and Proteomics

Of the n=52 subjects with a discharge MoCA score, n=23 had proteomic data for analysis. Likewise, of the n=28 subjects with a 90-day MoCA score, 13 had proteomic data for analysis. Table 3 demonstrates the top 6 most significant intracranial and systemic proteins correlated with discharge MoCA scores from n=23 subjects. Again, there were no significant relationships identified when assessing MoCA scores and patient sex, A1c, TSH, LDL, HDL, triglycerides, total cholesterol; diagnosis of hyperlipidemia, hypertension, diabetes; infarct time, infarct volume, atrial fibrillation or location/source of thrombus.

TABLE 3
Top 6 most significant intracranial and
systemic proteins related to discharge MoCA.
Proteins Significantly Correlated to Discharge MoCA Scores

| | | |
|---|---|---|
| Proteins | P-value | R² value |

Intracranial Samples

| | | |
|---|---|---|
| SERPINA7 | 0.01 | 0.28 |
| DNER | 0.01 | 0.27 |
| APOM | 0.02 | 0.24 |
| IGFBP3 | 0.03 | 0.21 |
| s-DPP4 | 0.05 | 0.18 |
| MEGF9 | 0.05 | 0.18 |

Systemic Samples

| | | |
|---|---|---|
| DNER | 0.001 | 0.42 |
| APOM | 0.004 | 0.33 |
| IGFBP3 | 0.008 | 0.29 |
| SCF | 0.02 | 0.22 |
| FAP | 0.03 | 0.20 |
| TGFBI | 0.03 | 0.20 |

n = 23 subjects. Proteins were ranked by smallest p-values and largest R² values; all correlations were positive

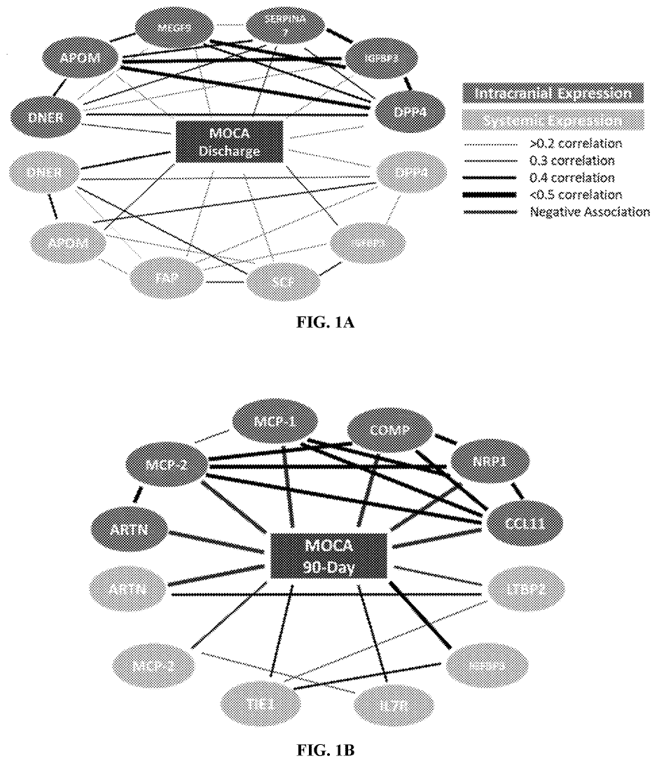

Most significant intracranial proteins correlated to discharge MoCA score (all with positive correlations) include thyroxine-binding globulin (SERPINA 7), delta and notch-like epidermal growth factor-related receptor (DNER), apolipoprotein M (APOM), insulin-like growth factor binding protein-3 (IGFBP3), soluble dipeptidyl peptidase-4 (s-DPP4), and multiple epidermal growth factor-like domains protein 9 (MEGF9). Most significant systemic proteins correlated to discharge MoCA score (all with positive correlations) include DNER, APOM, IGFBP3, stem cell factor (SCF), prolyl endopeptidase FAP (FAP), and transforming growth factor-beta-induced protein ig-h3 (TGFBI) (FIG. 1A-1B).

Both intracranial (dark grey) and systemic (light gray) findings are depicted in FIGS. 1A-1B and allow for proteomic comparisons across outcome measures. For example, several of the systemic proteins predictive of MoCA at discharge are also significant in the intracranial blood (DNER, APOM, IGFBP3, s-DPP4), indicating a similar response at the site of infarction compared to blood that could be sampled systemically. These proteomic webs demonstrate network strength (r2 value) and aids in the investigation of more complex protein-protein signaling pathways, rather than a singular protein at a specific timepoint.

Table 4 demonstrates the top 6 most significant intracranial and systemic proteins related to discharge MoCA scores from n=13 subjects. Proteins were ranked by smallest p-value and largest R² value. Most significant intracranial proteins correlated with 90-day MoCA (all were negative correlations) include artemin (ARTN), monocyte chemotactic protein-2 (MCP-2), monocyte chemotactic protein 1 (MCP-1), cartilage oligomeric matrix protein (COMP), neuropilin-1 (NRP1), and eotaxin (CCL11). Most significant systemic proteins negatively correlated with 90-day MoCA scores include ARTN, latent-transforming growth factor beta-binding protein 2 (LTBP2), and MCP-2. Most significant systemic proteins positively correlated with 90-day MoCA scores include insulin-like growth factor binding protein-3 (IGFBP3), tyrosine-protein kinase receptor tie-1 (TIE1), and interleukin-7 receptor subunit alpha (IL7R).

TABLE 4
Top 6 most significant intracranial and
systemic proteins related to 90-day MoCA.
Proteins Significantly Correlated to 90-day MoCA Scores

| | | |
|---|---|---|
| Proteins | P-value | R² value |

Intracranial Samples

| | | |
|---|---|---|
| ARTN* | 0.002 | 0.59 |
| MCP-2* | 0.003 | 0.57 |
| MCP-1* | 0.003 | 0.56 |
| COMP* | 0.007 | 0.46 |
| NRP1* | 0.007 | 0.49 |
| CCL11* | 0.01 | 0.46 |

Systemic Samples

| | | |
|---|---|---|
| ARTN* | 0.001 | 0.62 |
| IGFBP3 | 0.01 | 0.45 |
| LTBP2* | 0.02 | 0.43 |
| TIE1 | 0.02 | 0.43 |
| IL7R | 0.02 | 0.42 |
| MCP-2* | 0.02 | 0.42 |

Proteins were ranked by smallest p-values and largest R² values.
Asterisks indicate negative correlation while proteins without asterisks indicate positive correlations.

### Example 7: Discussion of Examples 2-6

Thrombectomy guidelines for ELVO stroke subjects have been generated by trials which measured neurologic/functional outcome, often the modified Rankin Score [34]. Systemic and intracranial proteomic data on ELVO subjects undergoing MT was used to identify proteomic biomarkers of cognition which may be both prognostic as well as targets for novel/existing therapies. Systemic blood is reliably accessible for potential prognostics while the analysis of intracranial blood reveals the local ischemic response, which identifies potential therapeutic targets.

Patient demographic data was investigated for predictors of cognitive performance. When assessing discharge MoCA scores, aside from basilar location of thrombus and LDL levels, there were no significant relationships with demographic/laboratory data nor infarct time. The finding of a positive correlation between LDL and discharge MoCA scores but not 90-day MoCA scores may be related to atherosclerotic burden at presentation; however, studies have reported few cognitive consequences related to chronic LDL levels [20]. When assessing 90-day MoCA scores, age was found to have a negative correlation, whereas having normal BMI was associated with lower cognitive scores. Both findings are unsurprising as age-related cognitive decline is well-known, and it has previously been reported that obese stroke patients are significantly younger (17 years) compared to the normal BMI cohort [17].

Next, systemic and intracranial proteins were investigated, which were found to have significant correlations to discharge MoCA scores as well as to 90-day MoCA scores. Interestingly, several of the proteins that were found to have a significant relationship with post-stroke cognitive function have been previously reported to play a role in stroke outcome and cognition/neurodegeneration.

First, the focus was on soluble dipeptidyl peptidase 4 (s-DPP4) and C-C motif chemokine 11 (CCL11) as biomarkers and potential therapeutics for post-stroke cognition that have been previously reported on in the context of stroke and in cognition. Soluble DPP-4 (s-DPP4) is well-known in the diabetes literature leading to the development of several inhibitors which help lower blood glucose levels. FDA-approved DPP4 inhibitors typically block the membrane bound form of DPP4, which increases s-DPP4. In the current study, the soluble form of DPP4 was studied and a positive correlation was found between intracranial s-DPP4 and discharge MoCA scores indicating higher s-DPP4 was predictive of better cognitive function. DPP4 inhibitors have previously been administered in several rodent models of stroke and have demonstrated efficacy in reducing injury and enhancing functional recovery [5]. Further, these inhibitors have been associated with improvement in cognition in a diabetic rat model and have been suggested as a potential treatment for Alzheimer's disease [1, 24]. Findings that increased s-DPP4 (a potential consequence of DPP4 inhibition), was predictive of better cognitive function corroborate prior findings in the stroke and cognition literature. In the study, intracranial CCL11 was also found to be negatively correlated with 90-day MoCA scores. This finding is unsurprising as CCL11 has been shown to be a causative factor in the cognitive decline of aging [35]. CCL11 is a ligand for the chemokine receptor type 3 (CCR3) receptor and, thus, CCR3 has been identified as a potential therapeutic target for Alzheimer's disease that reduces amyloid beta deposition and tau phosphorylation [35]. Interestingly, DPP4 has been shown to cleave CCL11 and reduce its chemotactic interaction with CCR3 [29]. Taking previous finding into the context of the current study, it was contemplated that s-DPP4 exerts a beneficial effect on cognition after ELVO by cleaving and inactivating chemokines such as CCL11 that impair cognition through the CCR3 receptor pathway. This supports existing literature that DPP4 inhibitors may be useful in combatting cognitive decline and offers a specific human pathology for future application.

Additional proteins which have been shown to be related to neurodegeneration include IGFBP3, DNER, and NRP1. Insulin-like growth factor-binding protein 3 (IGFBP3) is one of six members of a family known to carry IGF-1. In the study, intracranial IGFBP3 was found to be positively correlated to discharge MoCA score and similarly systemic IGFBP3 was positively correlated to both discharge and 90-day MoCA scores indicating higher IGFBP3 were predictive of better cognitive function. A prior study reported that low levels of IGFBP3 were predictive of worse functional outcome at one-year post-stroke based on modified Rankin scores [6]. The findings align with and add to this study by including cognitive function metrics after stroke. A separate study investigating insulin-like growth factors and cognitive function in the aging male population reported increased IGFBP3 was significantly associated with greater cognitive decline in their studied population [10]. Interestingly, the directionality of the findings are opposite to this study, which may offer a unique relationship between IGFBP3 and cognition in stroke patients specifically. Delta and Notch-like epidermal growth factor-related receptor (DNER) has been shown to activate the NOTCH1 pathway which has been reported to contribute to neurodegeneration and Alzheimer's pathophysiology [23]. In the study, both systemic and intracranial DNER were positively correlated with discharge MoCA scores but not at the later time point of 90-days, indicating a potential temporal change in the proteomic expression which may influence cognitive function in the sub-acute phase of recovery. Neuropilin-1 (NRP1) has been shown to be upregulated in patients with severe Alzheimer's disease [14]. One study reported NRP1 to interact with APOE-e4 in cognition as higher levels of NRP1 correlated to cognitive decline in patients with the APOE-e4 gene [21]. NRP1 has been shown to have a role in mitochondrial dysfunction, atherosclerosis, and neurodegeneration as well as in brain microvascular endothelial inflammation and blood-brain-barrier function [2, 32]. Not surprisingly, vascular dysfunction and blood-brain-barrier disruption have both been shown to be directly related to VCID [7]. In the cohort, NRP1 was found to be negatively correlated with 90-day MoCA scores indicating higher levels were associated with worse cognitive function as supported by prior literature.

Other proteins that stood out in the findings include MCP1 and COMP. A previously published meta-analysis reported increased circulating levels of monocyte chemotactic protein 1 (MCP1) was associated with increased long-term risk of stroke and that this protein may serve as a potential therapeutic target [9]. In the study, MCP1 and cognitive outcome after stroke were reported. Like the meta-analysis, increased MCP1 levels were found to be deleterious; specifically, intracranial MCP1 levels were negatively correlated with 90-day MoCA scores. A previous study reported cartilage oligomeric matrix protein (COMP) to be positively associated with worse plaque burden and plaques that were symptomatic in carotid atherosclerosis [11]. Again, the study adds to the existing literature by reporting on the relationship between COMP levels and cognitive function post-stroke. Like the prior study, COMP was found to be a negative factor; specifically, a negative correlation between intracranial COMP levels and 90-day MoCA scores is reported indicating higher COMP levels predicted worse cognitive function at the 90-day time point. As many ELVO strokes are atherosclerotic in etiology, the relationship between COMP and MoCA was found to be of particular interest.

Several biomarkers related to dementia are also linked to the development or prediction of stroke-induced dementia. Plasma levels of Ab42/40-b and ptau181 and total tau have been reported to be involved in the development of post-stroke cognitive impairment [3, 4, 31]. Both of these proteins are also associated with cerebral microbleeds, which is a risk factor for dementia. 32 Higher plasma NfL has been reported to be predictive of unfavorable functional outcomes after stroke. 33 GFAP has been reported to provide clinical information in differential diagnosis of different types of strokes https://doi.org/10.1007/BF03256432. These biomarker studies use patient data after the stroke and don't differentiate in the type of ischemic stroke. Additional studies are needed to determine if these biomarkers are present at the time of thrombectomy which is 3-12 h after the last known normal.

An existing limitation of BACTRAC is the geographic location where samples were collected. The study represents one population of the United States with limitations on diversity, mainly serving Caucasian individuals with homogenous comorbidities. However, a significant portion of the stroke patients are from rural areas of Appalachia, which represents a population with known health disparities. Access to this patient population will allow us to further study an underserved area where novel prognostics and therapeutic interventions would be greatly valued. Proteomic relationships with stroke outcomes in Appalachia will be the focus of subsequent studies. Another limitation of this study is the sample size of subjects with discharge and 90-day MoCA scores. The full MoCA is most appropriate for individuals with at most moderate impairment, as aphasia and other more severe impairments can interfere mask otherwise intact abilities (receptive language, verbal memory) on some items on the test. For such patients a Mini-MoCA better suited to the population was collected. There remains a potential selection effect, as only those with a MoCA or MiniMoCA score were included in the analysis, and thus the findings may be limited to those without profound post-stroke impairment. Data reported here will be validated as BACTRAC enrollment continues and larger analyses are conducted. Another constraint of this study is that it is limited to correlative analyses. For example, some proteins are elevated because of vascular injury and contribute to the injury, however, some proteins are consequentially upregulated as a response/protective/rescue measure. Further, some proteins may have high expression but lower activity or vice versa. However, these correlations still could serve as predictive biomarkers for cognitive performance after stroke. It is also important to emphasize these studies are directed to cognitive decline secondary to ELVO treated by MT, which is a very specific pathophysiology in a very specific cohort of patients. ELVO injury is significantly different from other types of stroke and small vessel disease and the cognitive decline after ELVO is likely different from cognitive decline secondary to dementias of varying etiologies. Lastly, proteins which have been shown to contribute to stroke severity focus on outcome metrics different from cognitive function tests, for example mRS. Here data is provided on systemic and intracranial protein expression in ELVO subjects treated with MT and how those proteins related to cognitive function at the time of discharge as well as at 90-day follow-up.

### Example 8: Analysis of Protein Network to Predict Cognitive Function Among Emergent Large Vessel Occlusion (ELVO) Patients Undergoing Mechanical Thrombectomy

The Blood And Clot Thrombectomy Registry And Collaboration (BACTRAC) study is a continually enrolling tissue bank (clinicaltrials.gov NCT03153683) and registry from stroke patients undergoing mechanical thrombectomy. Blood samples from systemic arterial blood (internal carotid artery) of twenty-three patients were collected and sent to Olink Proteomics (Olink Proteomics, Boston, Massachusetts, USA) for analysis of protein expression of 92 cardiometabolic proteins and 92 inflammatory proteins. To determine which proteins had the most significant changes based on MOCA scoring, a series of 184 paired t-tests were performed. Within each panel, proteins were then ranked based on the associated p values. Benjamini and Hochberg's linear step-up procedure was used to control the false discovery rate at 0.05. Pearson Correlation revealed proteins which were significantly related to the patients's discharge MOCA score as well as 90-day follow up MOCA. Data and network analyses were performed using IBM SPSS Statistics, SAS v 9.4, and STRING V11. Related to network of significant proteins, associated biological processes were identified from highest strength and least false discovery rate.

Twenty-two patients (15 with discharge day MiniMOCA and 7 with 90 day MOCA) were included in the analysis. Of these patients, 14 were females, and 9 were obese (BMI>30). 2 had minor stroke (NIHSS: 1-4), 13 had moderate stroke at admission (NIHSS: 5-15), 4 had moderate to severe stroke (NIHSS: 16-20), and 2 patients had severe stroke (NIHSS≥21). 19 patients had associated comorbidities (hypertension, diabetes, and hyperlipidemia). Mean last known normal to thrombectomy completion time was 621±333 minutes and mean infarct volume was 18,271±16,534 mm³. The 9 overlapping proteins from discharge to 90-day follow up were later analyzed using interconnected STRING to determine association network and proteomic biological functions. Nine proteins include: DPP4, NCAM1, TGFBI, PRCP, APOM, TIE1, QPCT, MEGF9, and IGFBP3. Biological processes relating to the network of 9 proteins are depicted in Table 5.

TABLE 5

| Biological Process | Strength | False discovery rate | Matching proteins in network (labels) |
|---|---|---|---|
| Protein metabolic | 0.59 | 1.32E−05 | ST8SIA4, ST8SIA2, TP53, IGF1, QPCT, |
| process | | | FN1, DPP4, TIE1, APOM, IGFBP3, PRCP, |
| | | | IGF2, GDNF, TGFBI, IGFALS, NCAM1 |
| Regulation of glucose | 1.61 | 0.0011 | IGF1, IGFBP3, GCG, IGF2 |
| metabolic process | | | |
| Cellular protein | 0.57 | 0.0011 | ST8SIA4, ST8SIA2, TP53, IGF1, QPCT, |
| metabolic process | | | FN1, TIE1, IGFBP3, IGF2, GDNF, TGFBI, |
| | | | IGFALS, NCAM1 |
| Positive regulation of | 1.93 | 0.0018 | IGF1, GCG, IGF2 |
| glucose metabolic | | | |
| process | | | |
| Regulation of | 2.84 | 0.0018 | GDNF, NCAM1 |
| semaphorin-plexin | | | |
| signaling pathway | | | |
| Regulation of protein | 1 | 0.0029 | TP53, IGF1, FN1, GIP, DPP4, GCG |
| transport | | | |
| Regulation of | 0.96 | 0.0032 | IGF1, FN1, GIP, DPP4, GCG, GDNF |
| secretion by cell | | | |
| Regulation of | 0.61 | 0.0037 | TP53, IGF1, FN1, GIP, DPP4, TIE1, |
| localization | | | IGFBP3, PRCP, GCG, GDNF |
| Ganglioside | 2.36 | 0.0038 | ST8SIA4, ST8SIA2 |
| biosynthetic process | | | |
| Regulation of cell | 0.53 | 0.0038 | TP53, IGF1, FN1, GIP, DPP4, IGFBP3, |
| communication | | | PRCP, GCG, IGF2, GDNF, NCAM1 |

### Example 9: Underserve Rural Patient Population

Work with the Kentucky Appalachian Stroke Registry (KApSR) identified factors contributing to regional health disparities. [37] This novel registry utilizes data from the UKY Stroke Care Network, an organization of stroke systems of care including 33 hospitals in Kentucky, Indiana, and West Virginia. Based on data from KApSR, 22% of the 8054 patients treated with cerebrovascular disease from 2010-2015 had hemorrhagic stroke [37], a notably higher proportion than the ~13% national average. Furthermore, over 78% of Appalachian stroke patients had 3 or more stroke-related comorbidities (i.e., multi-morbidities) irrespective of patient age [38].

Multi-morbidities and increased stroke rates in working-age individuals can be particularly devastating as they have a profound social impact on not only the patient who may themselves be a caregiver and/or wage earner, but also can affect their entire family and the community at large. In the under-resourced Appalachian population, multi-morbidities also identify those at greater risk for receiving suboptimal care and create barriers to long-term stroke rehabilitation. [38]

Considering the overall health of persons living in the Appalachian counties in Eastern Kentucky, it is difficult to ignore 1) the high rates of frequent mental distress (49th in the U.S.), 2) the socioeconomic disparities that contribute to an increased incidence of stroke in younger Appalachians (20-64 years old) [39], and 3) the high levels of environmental toxins, including radon and heavy metals [40], found in Appalachian communities.

Reviewing patient data in the BACTRAC database highlighted key issues including a large underserved population presenting with many of these comorbidities. Overall, low MOCA scores (worse) corresponded with high mRS and NIHSS scores (worse). MOCA scores did not significantly decline with age of the patient (p=0.2018). Smoking, weight, hypertension, diabetes, or hypercholesteremia did not individually predict cognitive performance. However, an increase in number of patient comorbidities was correlated to a decrease in cognition as depicted. These data demonstrates that multiple comorbidities associated with rural environments predict worse cognitive outcomes of stroke thrombectomy and led to development of a focus on rural-urban disparities.

### Example 10: BACTRAC Sampling

A key barrier to progress in stroke research has been a lack of translatable surrogates (e.g. in vitro cell cultures, animal models) to estimate, extrapolate, and model molecular events occurring within the human brain. To address this, the Blood and Clot Thrombectomy Registry and Collaboration (BACTRAC; NCT03153683) was developed. Through the standard thrombectomy process, the following is isolated: distal blood within the artery immediately downstream from the clot prior to its removal; peripheral blood just proximal to the clot (systemic arterial blood in the cervical feeding arteries to the brain); and the thrombus itself upon removal from the human subject [8].

### Example 11: DPP-4 Inhibitors for Treating VCID and Cognitive Recovery after Stroke Thrombectomy

Using BACTRAC data, machine learning and traditional statistical approaches were used to identify inflammatory cytokines and chemokines that predict edema and infarct volume. [33, 41] Linear regression and correlation analysis showed that time-of-stroke sDPP-4 levels positively correlated with performance on cognitive tests 90-days post-discharge. sDPP-4 is an ideal candidate to pursue as a biomarker for VCID, since elevated levels of this protein have been linked to improved cognition in other populations. [42] Moreover, the findings support considering sDPP-4 as a therapeutic target for VCID in that pharmacologically increasing its levels may diminish VCID through its signaling pathway inhibiting CCR3, which itself impairs cognition. Counterintuitively, FDA-approved DPP-4 inhibitors block the membrane bound form but increase soluble DPP-4. [43] These drugs are contemplated for use for treating VCID. The predictive method can be used target individuals that would require elevation in sDPP-4, who would be good candidates. It is contemplated that sDPP-4 cleaves chemokines to inactivate from stimulating anti-cognitive effects through the CCR3 receptor. DPP-4 inhibitors increase expression of sDPP-4 and thus elicit the pro-cognitive outcome.

Findings linking time-of-stroke sDDP-4 expression to cognition fits well with current knowledge of DPP-4 signaling. Catalytically active DPP-4 is released from the plasma membrane to produce circulating sDPP-4 (727 aa), which does not contain the intracellular tail and transmembrane regions and accounts for the majority of DPP-4 activity in human serum. [45] Soluble DPP-4 (sDPP-4) was initially identified in serum and saliva [46] and now found in cerebrospinal fluid, seminal fluid and bile. sDPP-4 activates intracellular signaling pathways that cause the proliferation of human lymphocytes, independent of either its catalytic activity [47] or the binding of ADA. [48] sDPP-4 inhibits insulin-mediated activation of Akt in cultured human adipocyte, skeletal muscle, and smooth muscle cells. [48] Remarkably, some of the actions attributed to sDPP-4, such as regulation of T-cell migration or up-regulation of costimulatory molecules require functional catalytic sDPP-4 catalytic activity [49, 50] Little is known on the mechanisms through which sDPP-4 activates signal transduction, possibly through interaction with the mannose 6-phosphate/IGF-2 receptor or via other molecular interactions. [51] Diabetes is the main pathology associated with DPP-4, so inhibitors were developed that lower high blood glucose levels to be used in the treatment of type 2 diabetes. [52] DPP-4 inhibitors slow the inactivation and degradation of GLP-1, a hormone involved in glucose removal from the gut. DPP-4 inhibitors improve blood glucose control and reduce both fasting and postprandial blood glucose levels, without causing weight gain. These inhibitors have been administered in several rodent models of stroke and have demonstrated efficacy in reducing injury and enhancing functional recovery. [53-55] Moreover, these therapeutics have been associated with improvement in cognition in a rat diabetic model [56] and have been suggested as a potential treatment for Alzheimer's disease. [57]

Downstream effects of sDPP-4 have been associate with cognition as well. The expression of CCR3 has been linked to age-related dementia. [58] In fact, antagonists to CCR3 have been shown to enhance cognitive performance in rodents. [59] As expected, ligands for CCR3, CCL5, CCL11 and CCL24, are also associated with increased expression during aging and cognitive deficits. [60-65] CCL5 has been reported to enhance vascular inflammation [66] and elevated expression of this chemokine is associated with cognitive decline. [67] The enzymatic activity of DPP-4 degrades a number of chemokines, which includes CCL5 and CCL11. Inhibitors of DPP-4 bind and inactivate the membrane form as a diabetes treatment but have been shown to increase levels of soluble DPP-4. These inhibitors have been linked to increased cognitive performance and the above reports suggest that elevated soluble DPP-4 levels degrade chemokines that activate CCR3 to reduce cognition during aging.

### Example 12: Other Proteins Associated with Cognitive Performance after ELVO

Using OLINK proteomics, expression of 184 proteins was measured from patient plasma at the time of stroke. sDDP-4, along with five other proteins, showed strong correlations with MOCA scores (cognition) at discharge. Additionally, when entered into a multiple linear regression model, the six proteins accounted for a significant amount of the variability in discharge MOCA scores (R2=0.66, p=. 006). Furthermore, there was no evidence that multicollinearity was a problem (largest variance inflation factor=2), suggesting that a larger sample will result in precise estimates of regression coefficients.

Delta and Notch-like epidermal growth factor-related receptor (DNER) is a proto-oncogene for hepatocellular carcinoma and a biomarker for poor prognosis. [68] It also is a transmembrane protein carrying extracellular EGF-like repeats and specifically expressed in somatodendritic regions of the cerebellum. [69] There do not appear to be any reports in the literature relating to stroke but this protein is highly expressed in substantia nigra and is studied as a potential biomarker for Parkinson Disease and Atypical Parkinson Syndrome. [70, 71] One study reports that DNER is increased in the plasma from traumatic brain injury patients. [72]

Cartilage acidic protein 1 (CRTAC1) is a glycosylated extracellular matrix protein which is secreted by chondrocytes and is associated with osteoarthritis. [74]. This protein is expressed in the brain and has been reported to be associated with neuronal plasticity after experimental stroke. [75]

Fibroblast activation protein (FAP) is a cell surface glycoprotein associated with wound healing, tissue remodeling, fibrosis, inflammation and tumor growth. [76] This transmembrane glycoprotein has dipeptidyl and endopeptidase activities and circulates in soluble cleaved form. [77] Patients with decrease in the activity of both DPP-4 and FAP during the first week after stroke onset had a more severe stroke and worse short-term outcomes.[78]

TNC Tenascin C (TNC), an extracellular matrix protein, regulates chemotaxis, phagocytosis and surveillance and proinflammatory cytokines of microglia after ischemic injury. [79] The expression of TNC has been correlated with unfavorable outcomes with endovascular thrombectomy. [80] The analysis shows that TNC expression and increased MOCA performance are inversely correlated suggesting TNC is impairing MOCA.

Macrophage inflammatory protein-3 (CCL20) is involved with autoimmune pathogenesis of the central nervous system and other organs. [81] This chemokine has been shown to increase its expression in traumatic brain injury and is directly neurotoxic to neurons and oligodendrocytes. [82] The neuroprotective signaling of human cord blood reduces CCL20 expression in a rat model of stroke. [83] Again, the analysis shows negative correlation with CCL20 expression and MOCA performance consistent with these reports.

These six proteins (CCL20, CRTAC1, DNER, DPP4, FAP, PTPRS, TNC) are significantly correlated with MOCA testing. CCL20 and TNC are inversely correlated, with MOCA indicating that a negative effect on cognitive recovery and these proteins are significantly correlated with each other. The others are significantly correlated with MOCA indicating a positive effect on cognitive recovery. Each one of these proteins including CCL20 and TNC have at least 3 significant correlations within this group. These results show that not only do these proteins show an association with cognitive outcome after thrombectomy but there are associations between them. This demonstrates a signaling network of both positive and negative effectors that are influencing cognitive recovery after thrombectomy.

Protein concentrations from individual patient samples were assessed with MoCA score at discharge and 90-days using Pearson correlation and Spearman correlation. Results are set forth in Table 6, with proteins identified with both measures in bold.

TABLE 6

| Protein- | | | Protein- | | |
|---|---|---|---|---|---|
| Pearson:MoCA | R-Value | P-Value | Spearman:MoCA | R-Value | P-Value |
| DNER | 0.668 | 0.000 | DNER | 0.701 | 0.000 |
| CRTAC1 | 0.638 | 0.001 | CRTAC1 | 0.693 | 0.000 |
| TGFBI | 0.499 | 0.015 | MEGF9 | 0.540 | 0.008 |
| DPP4 | 0.478 | 0.021 | DPP4 | 0.496 | 0.016 |
| LILRB5 | 0.476 | 0.022 | FAP | 0.485 | 0.019 |
| FAP | 0.474 | 0.022 | SCF | 0.475 | 0.022 |
| IGFBP3 | 0.433 | 0.039 | F7 | 0.457 | 0.028 |
| NCAM1 | 0.422 | 0.045 | PTPRS | 0.456 | 0.029 |
| PRCP | 0.422 | 0.045 | MFAP5 | 0.431 | 0.040 |
| PTPRS | 0.420 | 0.046 | CCL20 | −0.518 | 0.011 |
| IL4 | −0.483 | 0.020 | TNC | −0.518 | 0.011 |
| TNC | −0.466 | 0.025 | CCL23 | −0.502 | 0.015 |
| IL-1α | −0.454 | 0.030 | COL18A1 | −0.488 | 0.018 |
| CCL20 | −0.451 | 0.031 | ANG | −0.457 | 0.028 |
| | | | CCL14 | −0.439 | 0.036 |

### Example 13: ADRD Biomarkers Predictive of Dementia

Several biomarkers from systemic blood have been shown to be predictive of cognitive impairment. The University of Kentucky's Biomarker Center, under the directorship of Dr. Donna Wilcock, has been at the forefront of biomarker discovery predictive of cognitive impairment caused by AD and non-stroke VCID. The center's panel includes total tau, pTau181, Aβ₄₀, Aβ₄₂ (Aβ_{42/40}), GFAP, and NfL. Plasma levels of Aβ₄₂ and ptau181 and total tau have been reported to be involved in the development of post-stroke cognitive impairment. [3,4,31] Both of these proteins are also associated with cerebral microbleeds, which is a risk factor for dementia. [25] In addition, higher plasma NfL has been reported to be predictive of unfavorable functional outcomes after stroke. [84] These ADRD biomarkers demonstrate utility as predictors of post-stroke VCID. However, because stroke is an acute vascular event not always present in ADRD populations, the addition of other potential biomarkers could strengthen the predictive model for determining stroke patients most likely to incur cognitive impairment. The goal is to combine the findings with these markers of dementia with finding from the BACTRAC Study that has discovered several novel biomarkers associated with post-stroke cognitive impairment.

Example 14: BACTRAC Biomarker Study. Discovery of predictive biomarkers will permit the identification of patients most at risk for cognitive impairment. As shown above, the patient population is rural, and many live a far distance from a medical facility. Moreover, these biomarkers could detect hotspot areas for strokes associated with cognitive impairment. Using OLINK proteomics, expression of 184 proteins was measured from patient plasma obtained at the time of stroke. Six proteins showed strong correlations with MoCA scores (cognition) at hospital discharge. Additionally, when entered into a multiple linear regression model, these six proteins accounted for a significant amount of the variability in discharge MoCA scores (R2=0.66, p=. 006). There was no evidence of multicollinearity (largest variance inflation factor=2), suggesting that a larger sample will result in precise estimates of regression coefficients. Insulin Growth Factor Binding protein 3 (IGFBP3), Delta and notch-like epidermal growth factor (DNER), soluble DipeptidylPeptidase-4 (sDPP-4), Apolipoprotein M (APOM), Fibroblast Activation protein (FAP), and Stem Cell Factor (SCF) are top biomarker candidates for cognitive performance.

IGFBP3 is one of six members of this family known as a carrier of IGF-1. Its pleiotropic functions include binding to the proteins of the extracellular matrix and the proteins of the plasma membrane and translocating through the plasma membrane into the cytoplasm and into the nucleus, which implicate in many metabolic diseases, including cancer. [85] Levels of IGFBP3 reduce with aging and are associated with cognitive decline. [85] In the context of stroke, low blood levels of IGFBP3 within one of an ischemic stroke predict poor functional outcomes a year later. [87] In experimental models of stroke, inhibition of IGFBP3 enhanced injury while overexpression was protective. [88, 89] This protein not only shows correlation with MoCA at discharge but also at 90 days.

sDPP-4 is interesting for use in connection with VCID, since elevated levels of this protein have been linked to improved cognition in other populations. [84] Counterintuitively, FDA-approved DPP-4 inhibitors block the membrane-bound form of DPP-4 (mDPP-4) but increase soluble DPP-4 (sDPP-4). [43] It is contemplated that sDPP-4 exerts a beneficial effect on cognition by cleaving and inactivating chemokines (CCL5, CCL11, and CCL24) that impair cognition through the CCR3 receptor pathway. Diabetes is the main pathology associated with DPP-4, so inhibitors were developed that lower blood glucose levels, to treat type 2 diabetes. [52] These inhibitors have been administered in several rodent models of stroke and have demonstrated efficacy in reducing injury and enhancing functional recovery. [53-55] Moreover, these therapeutics have been associated with improvement in cognition in a rat diabetic model [56] and have been suggested as a potential treatment for Alzheimer's disease. [57] The expression of CCR3 has been linked to age-related dementia. [58] In fact, antagonists to CCR3 have been shown to enhance cognitive performance in rodents. As expected, ligands for CCR3, CCL5, CCL11, and CCL24 also show increased expression during aging and cognitive deficits. [60-65] CCL5 enhances vascular inflammation, [66] and elevated expression of this chemokine is associated with cognitive decline. [67] The increased expression of sDPP-4 in the plasma would decrease CCR3 signaling leading to enhanced cognition.

Delta and Notch-like epidermal growth factor-related receptor (DNER) is a proto-oncogene for hepatocellular carcinoma and a biomarker for poor prognosis. [68] It is a transmembrane protein carrying extracellular EGF-like repeats, specifically expressed in somatodendritic regions of the cerebellum. [69] There do not appear to be any reports relating to stroke, but this protein is highly expressed in substantia nigra and is studied as a potential biomarker for Parkinson's disease and atypical Parkinson syndrome. [70, 71] One study reported that DNER is increased in the plasma of traumatic brain injury patients.[72]

APOM is apolipoprotein M, which is critical in the formation of high-density lipoprotein and cholesterol efflux to HDL. Gene polymorphisms are associated with increased ischemic stroke in Han Chinese [90]. In addition, plasma APOM is negatively correlated with acute myocardial infarction [91], and this protein has been used in an experimental therapeutic strategy to treat stroke [92].

Fibroblast activation protein (FAP) is a cell surface glycoprotein associated with wound healing, tissue remodeling, fibrosis, inflammation and tumor growth. [93] This transmembrane glycoprotein has dipeptidyl and endopeptidase activities and circulates in soluble cleaved form. [94] Patients with a decrease in the activity of both DPP4 and FAP during the first week after stroke onset had a more severe stroke and worse short-term outcomes.[95]

Stem Cell Factor (SCF) is a ligand for c-kit, which is a receptor for a number of cell types, including hematopoietic stem cells, mast cells, melanocytes, and germ cells, and has been implicated in a number of human cancers. [96] Increased expression of SCF is linked to shorter survival time in patients with malignant gliomas. [97] SCF in combination with granulocyte-colony stimulating factor has been reported to improve functional recovery after chronic stroke. [98] This combination has demonstrated efficacy in aged rodents and can be used weeks to months after stroke has occurred. [99] After administration of this combination, one marrow-derived endothelial cells are increased in brains of mice following ischemic stroke, which are perceived to repair the injured tissue. [100] This hematopoietic cytokine has been proposed to be a treatment for spinal cord injury due to its protective effect on spinal neurons. [101]

TABLE 7
Protein Correlation with MoCA Score

| Protein | R-Value | P-Value |
|---|---|---|
| DNER | 0.6449 | 0.0008 |
| APOM | 0.5749 | 0.0041 |
| IGFBP3 | 0.537 | 0.0082 |
| SCF | 0.4725 | 0.0228 |
| FAP | 0.4506 | 0.0309 |
| sDPP4 | 0.4438 | 0.0339 |

With reference to Table 7, these six proteins are significantly correlated with MoCA testing of cognition at hospital discharge indicating a positive effect on cognitive recovery. Each one of these proteins has at least 3 significant correlations within this group. These results demonstrate that not only do these proteins associate with cognitive outcomes after thrombectomy, but they show associations among themselves as well. These results also demonstrate a signaling network of positive effectors that influence cognitive recovery after thrombectomy. Thus, the statistical analyses and literature support that this proteomic network represents a group of potential predictors of cognitive outcomes after large vessel occlusion. However, the increased sample may shift its significance in the framework of predicting cognitive outcomes.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Angelopoulou E, Piperi C. DPP-4 inhibitors: a promising therapeutic approach against Alzheimer's disease. Ann Transl Med. 2018; 6:255.
2. Bosseboeuf E, Raimondi C. Signalling, metabolic pathways and iron homeostasis in endothelial cells in health, atherosclerosis and Alzheimer's disease. Cells. 2020; 9:1-39.
3. Chen H, Gu S, Liu X, Xie A, Wang C. Association of blood amyloid beta-protein 1-42 with poststroke cognitive impairment: a systematic review and meta-analysis. Biomed Res Int. 2022; 2022:6552781.
4. Chi N F, Chao S P, Huang L K, Chan L, Chen Y R, Chiou H Y, et al. Plasma amyloid beta and tau levels are predictors of post-stroke cognitive impairment: a longitudinal study. Front Neurol. 2019; 10:715.
5. Chiazza F, Tammen H, Pintana H, Lietzau G, Collino M, Nystrom T, et al. The effect of DPP-4 inhibition to improve functional outcome after stroke is mediated by the SDF-1alpha/CXCR4 pathway. Cardiovasc Diabetol. 2018; 17:60.
6. Ebinger M, Ipsen N, Leonards C O, Empl L, Hanne L, Liman T, et al. Circulating insulin-like growth factor binding protein-3 predicts one-year outcome after ischemic stroke. Exp Clin Endocrinol Diabetes. 2015; 123:461-5.
7. Frantellizzi V, Pani A, Ricci M, Locuratolo N, Fattapposta F, De Vincentis G. Neuroimaging in vascular cognitive impairment and dementia: a systematic review. J Alzheimers Dis. 2020; 73:1279-94.
8. Fraser J F, Collier L A, Gorman A A, Martha S R, Salmeron K E, Trout A L, et al. The Blood And Clot Thrombectomy Registry And Collaboration (BACTRAC) protocol: novel method for evaluating human stroke. J Neurointerv Surg. 2018; 11:265-70.
9. Georgakis M K, Malik R, Bjorkbacka H, Pana T A, Demissie S, Ayers C, et al. Circulating monocyte chemoattractant protein-1 and risk of stroke: meta-analysis of population-based studies involving 17 180 individuals. Circ Res. 2019; 125:773-82.
10. Green C J, Holly J M, Bayer A, Fish M, Ebrahim S, Gallacher J, et al. The role of IGF-I, IGF-II, and IGFBP-3 in male cognitive aging and dementia risk: the Caerphilly prospective study. J Alzheimers Dis. 2014; 41:867-75.
11. Hultman K, Edsfeldt A, Bjorkbacka H, Duner P, Sundius L, Nitulescu M, et al. Cartilage oligomeric matrix protein associates with a vulnerable plaque phenotype in human atherosclerotic plaques. Stroke. 2019; 50:3289-92.
12. Kalaria R N, Akinyemi R, Ihara M. Stroke injury, cognitive impairment and vascular dementia. Biochim Biophys Acta. 1862; 915-925:2016.
13. Lewczuk P, Lukaszewicz-Zajac M, Mroczko P, Kornhuber J. Clinical significance of fluid biomarkers in Alzheimer's Disease. Pharmacol Rep. 2020; 72:528-42.
14. Lim K H, Yang S, Kim S H, Joo J Y. Identifying New COVID-19 receptor neuropilin-1 in severe Alzheimer's disease patients group brain using genome-wide association study approach. Front Genet. 2021; 12:741175.
15. Lopez-Cancio E, Jovin T G, Cobo E, Cerda N, Jimenez M, Gomis M, et al. Endovascular treatment improves cognition after stroke: a secondary analysis of REVASCAT trial. Neurology. 2017; 88:245-51.
16. Maglinger B, Frank J A, McLouth C J, Trout A L, Roberts J M, Grupke S, et al. Proteomic changes in intracranial blood during human ischemic stroke. J Neurointerv Surg. 2021; 13 (4): 395-9.
17. Maglinger B, McLouth C J, Frank J A, Rupareliya C, Sands M, Sheikhi L, et al. Influence of BMI on adenosine deaminase and stroke outcomes in mechanical thrombectomy subjects. Brain Behav Immun Health. 2022; 20:100422.
18. Maglinger B, Sands M, Frank J A, McLouth C J, Trout A L, Roberts J M, et al. Intracranial VCAM1 at time of mechanical thrombectomy predicts ischemic stroke severity. J Neuroinflammation. 2021; 18:109.
19. Malhotra K, Gornbein J, Saver J L. Ischemic strokes due to large-vessel occlusions contribute disproportionately to stroke-related dependence and death: a review. Front Neurol. 2017; 8:651.
20. Mefford M T, Chen L, Lewis C E, Muntner P, Sidney S, Launer L J, et al. Long-term levels of LDL-C and cognitive function: The CARDIA Study. J Int Neuropsychol Soc. 2021; 27:1048-57.
21. Moore A M, Mahoney E, Dumitrescu L, De Jager P L, Koran M E I, Petyuk V A, et al. APOE epsilon4-specific associations of VEGF gene family expression with cognitive aging and Alzheimer's disease. Neurobiol Aging. 2020; 87:18-25.
22. Paolucci S, Antonucci G, Gialloreti L E, Traballesi M, Lubich S, Pratesi L, et al. Predicting stroke inpatient rehabilitation outcome: the prominent role of neuropsychological disorders. Eur Neurol. 1996; 36:385-90.
23. Perna A, Marathe S, Dreos R, Falquet L, Akarsu Egger H, Auber L A. Revealing NOTCH-dependencies in synaptic targets associated with Alzheimer's disease. Mol Cell Neurosci. 2021; 115:103657.
24. Pintana H, Apaijai N, Chattipakorn N, Chattipakorn S C. DPP-4 inhibitors improve cognition and brain mitochondrial function of insulin-resistant rats. J Endocrinol. 2013; 218:1-11.
25. Romero J R, Demissie S, Beiser A, Himali J J, DeCarli C, Levy D, et al. Relation of plasma beta-amyloid, clusterin, and tau with cerebral microbleeds: Framingham heart study. Ann Clin Transl Neurol. 2020; 7:1083-91.
26. Sands M, Frank J A, Maglinger B, McLouth C J, Trout A L, Turchan-Cholewo J, et al. Antimicrobial protein REG3A and signaling networks are predictive of stroke outcomes. J Neurochem. 2022; 160 (1): 100-12.
27. Saxena S K, Ng T P, Koh G, Yong D, Fong N P. Is improvement in impaired cognition and depressive symptoms in post-stroke patients associated with recovery in activities of daily living? Acta Neurol Scand. 2007; 115:339-46.
28. Strambo D, Bartolini B, Beaud V, Marto J P, Sirimarco G, Dunet V, et al. Thrombectomy and thrombolysis of isolated posterior cerebral artery occlusion: cognitive, visual, and disability outcomes. Stroke. 2020; 51:254-61.
29. Struyf S, Proost P, Schols D, De Clercq E, Opdenakker G, Lenaerts J P, et al. CD26/dipeptidyl-peptidase IV down-regulates the eosinophil chemotactic potency, but not the anti-HIV activity of human eotaxin by affecting its interaction with CC chemokine receptor 3. J Immunol. 1999; 162:4903-9.
30. Tatemichi T K, Desmond D W, Stern Y, Paik M, Sano M, Bagiella E. Cognitive impairment after stroke: frequency, patterns, and relationship to functional abilities. J Neurol Neurosurg Psychiatry. 1994; 57:202-7.
31. Thomas A J, Hamilton C A, Heslegrave A, Barker S, Durcan R, Lawley S, et al. A longitudinal study of plasma pTau181 in mild cognitive impairment with Lewy bodies and Alzheimer's disease. Mov Disord. 2022; 37:1495-504.
32. Wang Y, Cao Y, Mangalam A K, Guo Y, LaFrance-Corey R G, Gamez J D, et al. Neuropilin-1 modulates interferon-gamma-stimulated signaling in brain microvascular endothelial cells. J Cell Sci. 2016; 129:3911-21.
33. Xu G, Dong X, Niu X, Zheng G, Wang H, Zhang F, et al. Cognitive function and prognosis of multimodal neuroimage-guided thrombectomy on mild to moderate anterior circulation infarction patients with broadened therapeutic window: a prospective study. Eur Neurol. 2017; 78:257-63.
34. Yaeger K A, Martini M L, Hardigan T, Ladner T, Hao Q, Singh I P, et al. Mortality reduction after thrombectomy for acute intracranial large vessel occlusion: meta-analysis of randomized trials. J Neurointerv Surg. 2020; 12:568-73.
35. Zhu C, Xu B, Sun X, Zhu Q, Sui Y. Targeting CCR3 to reduce amyloid-beta production, tau hyperphosphorylation, and synaptic loss in a mouse model of Alzheimer's disease. Mol Neurobiol. 2017; 54:7964-78.
36. Maglinger, B., Harp, J. P., Frank, J. A. et al. Inflammatory-associated proteomic predictors of cognitive outcome in subjects with ELVO treated by mechanical thrombectomy. BMC Neurol 23, 214 (2023).
37. Kitzman, P., et al., The Kentucky Appalachian Stroke Registry (KApSR). J Stroke Cerebrovasc Dis, 2018. 27 (4): p. 900-907.
38. Kitzman, P. H., et al., The Prevalence of Multiple Comorbidities in Stroke Survivors in Rural Appalachia and the Clinical Care Implications. J Stroke Cerebrovasc Dis, 2019. 28 (11): p. 104358.
39. Gehefer, G. O., B. M. White, and K. N. Simpson, Admissions for Ischemic Stroke Among Younger Adults in Kentucky: A Comparison of Appalachian and Non-Appalachian Counties. J Allied Health, 2019. 48 (2): p. e61-e64.
40. Rodriguez, S. D., et al., A Social-Ecological Review of Cancer Disparities in Kentucky. South Med J, 2018. 111 (4): p. 213-219.
41. Kleine, J. F., et al., Discrepancy between early neurological course and mid-term outcome in older stroke patients after mechanical thrombectomy. J Neurointerv Surg, 2016. 8 (7): p. 671-6.
42. Ohnuma, K., et al., Soluble CD26/dipeptidyl peptidase IV induces T cell proliferation through CD86 up-regulation on APCs. J Immunol, 2001. 167 (12): p. 6745-55.
43. Varin, E. M., et al., Circulating Levels of Soluble Dipeptidyl Peptidase-4 Are Dissociated from Inflammation and Induced by Enzymatic DPP4 Inhibition. Cell Metab, 2019. 29 (2): p. 320-334 e5.
44. Durinx, C., et al., Molecular characterization of dipeptidyl peptidase activity in serum: soluble CD26/dipeptidyl peptidase IV is responsible for the release of X-Pro dipeptides. Eur J Biochem, 2000. 267 (17): p. 5608-13.
45. Nagatsu, I., T. Nagatsu, and T. Yamamoto, Hydrolysis of amino acid beta-naphthylamides by aminopeptidases in human parotid salva and human serum. Experientia, 1968. 24 (4): p. 347-8.
46. Cordero, O. J., F. J. Salgado, and M. Nogueira, On the origin of serum CD26 and its altered concentration in cancer patients. Cancer Immunol Immunother, 2009. 58 (11): p. 1723-47.
47. Yu, D. M., et al., Soluble CD26/dipeptidyl peptidase IV enhances human lymphocyte proliferation in vitro independent of dipeptidyl peptidase enzyme activity and adenosine deaminase binding. Scand J Immunol, 2011. 73 (2): p. 102-11.
48. Lamers, D., et al., Dipeptidyl peptidase 4 is a novel adipokine potentially linking obesity to the metabolic syndrome. Diabetes, 2011. 60 (7): p. 1917-25.
49. Ohnuma, K., et al., Soluble CD26/dipeptidyl peptidase IV induces T cell proliferation through CD86 up-regulation on APCs. J Immunol, 2001. 167 (12): p. 6745-55.
50. Ikushima, H., et al., Soluble CD26/dipeptidyl peptidase IV enhances transendothelial migration via its interaction with mannose 6-phosphate/insulin-like growth factor II receptor. Cell Immunol, 2002. 215 (1): p. 106-10.
51. Zhong, J., X. Rao, and S. Rajagopalan, An emerging role of dipeptidyl peptidase 4 (DPP4) beyond glucose control: potential implications in cardiovascular disease. Atherosclerosis, 2013. 226 (2): p. 305-14.
52. Rohrborn, D., N. Wronkowitz, and J. Eckel, DPP4 in Diabetes. Front Immunol, 2015. 6: p. 386.
53. Darsalia, V., et al., The DPP-4 inhibitor linagliptin counteracts stroke in the normal and diabetic mouse brain: a comparison with glimepiride. Diabetes, 2013. 62 (4): p. 1289-96.
54. Chiazza, F., et al., The effect of DPP-4 inhibition to improve functional outcome after stroke is mediated by the SDF-1alpha/CXCR4 pathway. Cardiovasc Diabetol, 2018. 17 (1): p. 60.
55. Ma, M., et al., DPP-4 inhibition with linagliptin ameliorates cognitive impairment and brain atrophy induced by transient cerebral ischemia in type 2 diabetic mice. Cardiovasc Diabetol, 2015. 14: p. 54.
56. Pintana, H., et al., DPP-4 inhibitors improve cognition and brain mitochondrial function of insulin-resistant rats. J Endocrinol, 2013. 218 (1): p. 1-11.
57. Angelopoulou, E. and C. Piperi, DPP-4 inhibitors: a promising therapeutic approach against Alzheimer's disease. Ann Transl Med, 2018. 6 (12): p. 255.
58. Zhu, C., et al., Targeting CCR3 to Reduce Amyloid-beta Production, Tau Hyperphosphorylation, and Synaptic Loss in a Mouse Model of Alzheimer's Disease. Mol Neurobiol, 2017. 54 (10): p. 7964-7978.
59. Sui, Y., et al., The small molecular CCR3 antagonist YM344031 attenuates neurodegenerative pathologies and improves learning and memory performance in a mouse model of Alzheimer's disease. Brain Res, 2019. 1719: p. 1-10.
60. Huber, A. K., et al., An emerging role for eotaxins in neurodegenerative disease. Clin Immunol, 2018. 189: p. 29-33.
61. Bettcher, B. M., et al., MCP-1 and eotaxin-1 selectively and negatively associate with memory in MCI and Alzheimer's disease dementia phenotypes. Alzheimers Dement (Amst), 2016. 3: p. 91-7.
62. Butcher, L., et al., Association between plasma CCL11 (eotaxin-1) and cognitive status in older adults: Differences between rural and urban dwellers. Exp Gerontol, 2018. 113: p. 173-179.
63. Sirivichayakul, S., et al., Eotaxin, an Endogenous Cognitive Deteriorating Chemokine (ECDC), Is a Major Contributor to Cognitive Decline in Normal People and to Executive, Memory, and Sustained Attention Deficits, Formal Thought Disorders, and Psychopathology in Schizophrenia Patients. Neurotox Res, 2019. 35 (1): p. 122-138.
64. Stamatovich, S. N., et al., Plasma pro-and anti-inflammatory cytokines may relate to cocaine use, cognitive functioning, and depressive symptoms in cocaine use disorder. The American Journal of Drug and Alcohol Abuse, 2021. 47 (1): p. 52-64.
65. Teixeira, A. L., et al., Revisiting the Role of Eotaxin-1/CCL11 in Psychiatric Disorders. Front Psychiatry, 2018. 9: p. 241.
66. Louboutin, J. P., et al., Role of CCR5 and its ligands in the control of vascular inflammation and leukocyte recruitment required for acute excitotoxic seizure induction and neural damage. FASEB J, 2011. 25 (2): p. 737-53.
67. Poletti, S., et al., Circulating inflammatory markers impact cognitive functions in bipolar depression. J Psychiatr Res, 2021. 140: p. 110-116.
68. Liang, Y., et al., Oncogene Delta/Notch-Like EGF-Related Receptor Promotes Cell Proliferation, Invasion, and Migration in Hepatocellular Carcinoma and Predicts a Poor Prognosis. Cancer Biother Radiopharm, 2018. 33 (9): p. 380-386.
69. Saito, S. Y. and H. Takeshima, DNER as key molecule for cerebellar maturation. Cerebellum, 2006. 5 (3): p. 227-31.
70. Santaella, A., et al., Inflammation biomarker discovery in Parkinson's disease and atypical parkinsonisms. BMC Neurol, 2020. 20 (1): p. 26.
71. Jabbari, E., et al., Proximity extension assay testing reveals novel diagnostic biomarkers of atypical parkinsonian syndromes. J Neurol Neurosurg Psychiatry, 2019. 90 (7): p. 768-773.
72. Dyhrfort, P., et al., Monitoring of Protein Biomarkers of Inflammation in Human Traumatic Brain Injury Using Microdialysis and Proximity Extension Assay Technology in Neurointensive Care. J Neurotrauma, 2019. 36 (20): p. 2872-2885.
73. Steck, E., et al., Chondrocyte secreted CRTAC1: a glycosylated extracellular matrix molecule of human articular cartilage. Matrix Biol, 2007. 26 (1): p. 30-41.
74. Styrkarsdottir, U., et al., The CRTAC1 protein in plasma associates with osteoarthritis and predicts progression to joint replacements: a large-scale proteomics scan in Iceland. Arthritis Rheumatol, 2021.
75. Takase, H., et al., LOTUS overexpression accelerates neuronal plasticity after focal brain ischemia in mice. PLOS One, 2017. 12 (9): p. e0184258.
76. Hamson, E. J., et al., Understanding fibroblast activation protein (FAP): substrates, activities, expression and targeting for cancer therapy. Proteomics Clin Appl, 2014. 8 (5-6): p. 454-63.
77. Collins, P. J., et al., Purification, identification and characterisation of seprase from bovine serum. Int J Biochem Cell Biol, 2004. 36 (11): p. 2320-33.
78. Baerts, L., et al., Acute Ischemic Stroke Severity, Progression, and Outcome Relate to Changes in Dipeptidyl Peptidase IV and Fibroblast Activation Protein Activity. Transl Stroke Res, 2017. 8 (2): p. 157-164.
79. Manrique-Castano, D., et al., Tenascin-C preserves microglia surveillance and restricts leukocyte and, more specifically, T cell infiltration of the ischemic brain. Brain Behav Immun, 2021. 91: p. 639-648.
80. Zang, N., et al., Biomarkers of Unfavorable Outcome in Acute Ischemic Stroke Patients with Successful Recanalization by Endovascular Thrombectomy. Cerebrovasc Dis, 2020. 49 (6): p. 583-592.
81. Meitei, H. T., N. Jadhav, and G. Lal, CCR6-CCL20 axis as a therapeutic target for autoimmune diseases. Autoimmun Rev, 2021. 20 (7): p. 102846.
82. Leonardo, C. C., et al., CCL20 is associated with neurodegeneration following experimental traumatic brain injury and promotes cellular toxicity in vitro. Translational Stroke Research, 2012. 3 (3): p. 357-363.
83. Shahaduzzaman, M., et al., Human umbilical cord blood cells induce neuroprotective change in gene expression profile in neurons afteriIschemia through activation of Akt pathway. Cell Transplant, 2015.
84. Lewczuk, P., et al., Clinical significance of fluid biomarkers in Alzheimer's Disease. Pharmacol Rep, 2020. 72 (3): p. 528-542.
85. Varma Shrivastav, S., et al., Insulin-Like Growth Factor Binding Protein-3 (IGFBP-3): Unraveling the Role in Mediating IGF-Independent Effects Within the Cell. Front Cell Dev Biol, 2020. 8: p. 286.
86. Green, C. J., et al., The role of IGF-I, IGF-II, and IGFBP-3 in male cognitive aging and dementia risk: the Caerphilly Prospective Study. J Alzheimers Dis, 2014. 41 (3): p. 867-75.
87. Ebinger, M., et al., Circulating insulin-like growth factor binding protein-3 predicts one-year outcome after ischemic stroke. Exp Clin Endocrinol Diabetes, 2015. 123 (8): p. 461-5.
88. Song, B., et al., MiR-125a-5p silencing inhibits cerebral ischemia-induced injury through targeting IGFBP3. Folia Neuropathol, 2021. 59 (2): p. 121-130.
89. Chai, Z., et al., Inhibition of miR-19a-3p decreases cerebral ischemia/reperfusion injury by targeting IGFBP3 in vivo and in vitro. Biol Res, 2020. 53 (1): p. 17.
90. Zhao, D., et al., Association of apolipoprotein M gene polymorphisms with ischemic stroke in a Han Chinese population. J Mol Neurosci, 2011. 43 (3): p. 370-5.
91. Borup, A., et al., Apolipoprotein M in lipid metabolism and cardiometabolic diseases. Curr Opin Lipidol, 2015. 26 (1): p. 48-55.
92. Swendeman, S. L., et al., An engineered SIP chaperone attenuates hypertension and ischemic injury. Sci Signal, 2017. 10 (492).
93. Hamson, E. J., et al., Understanding fibroblast activation protein (FAP): substrates, activities, expression and targeting for cancer therapy. Proteomics Clin Appl, 2014. 8 (5-6): p. 454-63.
94. Collins, P. J., et al., Purification, identification and characterisation of seprase from bovine serum. Int J Biochem Cell Biol, 2004. 36 (11): p. 2320-33.
95. Baerts, L., et al., Acute Ischemic Stroke Severity, Progression, and Outcome Relate to Changes in Dipeptidyl Peptidase IV and Fibroblast Activation Protein Activity. Transl Stroke Res, 2017. 8 (2): p. 157-164.
96. Ronnstrand, L., Signal transduction via the stem cell factor receptor/c-Kit. Cell Mol Life Sci, 2004. 61 (19-20): p. 2535-48.
97. Sun, L., et al., Neuronal and glioma-derived stem cell factor induces angiogenesis within the brain. Cancer Cell, 2006. 9 (4): p. 287-300.
98. Piao, C. S., et al., The role of stem cell factor and granulocyte-colony stimulating factor in brain repair during chronic stroke. J Cereb Blood Flow Metab, 2009. 29 (4): p. 759-70.
99. Piao, C. S., et al., The combination of stem cell factor and granulocyte-colony stimulating factor for chronic stroke treatment in aged animals. Exp Transl Stroke Med, 2012. 4 (1): p. 25.
100. Toth, Z. E., et al., The combination of granulocyte colony-stimulating factor and stem cell factor significantly increases the number of bone marrow-derived endothelial cells in brains of mice following cerebral ischemia. Blood, 2008. 111 (12): p. 5544-52.
101. Yamasaki, K., et al., Stem cell factor prevents neuronal cell apoptosis after acute spinal cord injury. Spine (Phila Pa 1976), 2009. 34 (4): p. 323-7.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of predicting stroke-induced cognitive impairment in a subject, comprising:
a) obtaining, prior to recanalization, an intracranial blood sample from the subject and a systemic blood sample from the subject;
b) detecting target proteins in the samples, wherein the target proteins are selected from the group set forth in Table 1;
c) predicting a negative effect on cognitive recovery when the target proteins are detected in one or both of the sample.

2. The method of claim 1, and further comprising administering a treatment for cognitive impairment to the subject, when a negative effect on cognitive recovery is predicted.

3. The method of claim 2, wherein the subject has emergent large vessel occlusion (ELVO).

4. The method of claim 2, wherein the treatment comprises sDPP-4 or a DPP-4 inhibitor.

5. The method of claim 4, wherein the DPP-4 inhibitor is a gliptin, linagliptin, sitagliptin, vildagliptin, saxagliptin, gemigliptin, anagliptin, teneligliptin, alogliptin, trelagliptin, omarigliptin, evogliptin, gosogliptin, dutogliptin, or berberine,

6. The method of claim 1, and further comprising isolating plasma from the sample for use in detecting the target proteins.

7. The method of claim 6, wherein the subject is a stroke patient who has undergone mechanical thrombectomy (MT).

8. The method of claim 7, wherein the blood sample is peripheral blood collected just proximal to a thrombus removed by the MT.

9. The method of claim 7, and further comprising obtaining a proximal sample comprising blood collected proximal to a thrombus in the subject, and a distal sample comprising blood collected distal to the thrombus in the subject.

10. The method of claim 1, and further comprising obtaining another blood sample from the subject, collected at a distinct time.

11. The method of claim 10, and further comprising comparing the detected target proteins in the sample to the detected target proteins in the second sample.

12. A device for use in identifying risk of stroke-induced cognitive impairment in a subject, comprising: a combination of probes affixed to a substrate, comprising a probe specific for each of the target proteins, wherein the target proteins are selected from the group set forth in Table 1.

13. The device of claim 12, provided as a microfluidic enzyme-linked immunosorbent assay (ELISA) device.

14. A method of improving cognitive recovery for a subject undergoing a mechanical thrombectomy procedure, comprising:
administering to the subject an effective amount of a sDPP-4 or a DPP-4 inhibitor.

15. The method of claim 14, wherein the subject has emergent large vessel occlusion (ELVO).

16. The method of claim 14, and further comprising obtaining a blood sample from the subject and detecting target proteins in the sample, wherein the target proteins are selected from the group set forth in Table 1.
